(19) United States
(12) Patent Application Publication
(10) Pub. No.: US 2011/0137178 A1
(43) Pub. Date: Jun. 9, 2011

(54) Devices and methods for imaging particular cells including eosinophils

(75) Inventors: Guillermo J. Tearney, Cambridge, MA (US); Brett E. Bouma, Quincy, MA (US); Dongkyun Kang, Somerville, MA (US); Hongki Yoo, Melrose, MA (US)

(73) Assignee: The General Hospital Corporation, Boston, MA (US)

(21) Appl. No.: 12/899,524

(22) Filed: Oct. 6, 2010

Related U.S. Application Data
(60) Provisional application No. 61/249,207, filed on Oct. 6, 2009.

(51) Int. Cl. A61B 6/00

(52) U.S. Cl. 600/476

(57) ABSTRACT

An exemplary embodiment of apparatus and method according to the present disclosure can be provided. For example, using at least one first arrangement, it is possible to direct at least one first electro-magnetic radiation to at least one portion of tissue within a body. Using at least one second arrangement, it is possible to receive at least one second electro-magnetic radiation provided from the portion, which is based on the first electro-magnetic radiation. Further, using at least one third arrangement, it is possible to differentiate at least one particular cell which is eosinophil, mast cell, basophil, monocyte and/or nutrophil from other cells in the portion based on the second electro-magnetic radiation.

### CROSS-REFERENCE TO RELATED APPLICATION(S)

This application is based upon and claims the benefit of priority from U.S. Patent Application Ser. No. 61/249,207, filed on Oct. 6, 2009, the entire disclosure of which is incorporated herein by reference.

### FIELD OF THE DISCLOSURE

The present disclosure relates to exemplary embodiments of apparatus and methods for imaging particular cells, including but not limited to eosinophils, and more particularly to such exemplary apparatus and methods which can use, e.g., confocal microscopy to image particular cells including eosinophils.

### BACKGROUND INFORMATION

### Overview

Eosinophilic esophagitis (EoE) is a disease that afflicts both children and adults, thought to be caused by food allergy, and characterized by the presence of eosinophils within the esophageal squamous epithelium. EoE is now known to be common and rapidly increasing in incidence. EoE patients can experience symptoms ranging from nausea and vomiting to dysphagia and food impaction. It is generally thought that if left untreated, the eosinophilic inflammation may lead to significant and permanent damage, including fibrosis of the lamina propria, strictures, and perforations. As a result, EoE patients are currently treated until both symptoms and esophageal eosinophils abate.

While corticosteroids are effective for EoE, the disease usually returns when the drug is discontinued. The other therapy for EoE is aggressive dietary restriction. Once on a restricted diet, foods are gradually reintroduced until the diet becomes acceptable or symptoms and/or esophageal eosinophils recur. Since the only objective method for diagnosing EoE is histopathologic assessment of multiple upper endoscopic biopsies, monitoring the esophagus during therapy and the food reintroduction process involves a high number of repeat endoscopies. This process is time consuming and frustrating for patients and their families. Because endoscopic biopsy requires conscious sedation, it can also be costly, placing a substantial financial burden on our health care system. Given the rapid increase in the number of patients with this disease, there is a compelling need for a less invasive and more cost effective ways for identifying eosinophils in the esophagus.

Confocal microscopy has been developed for imaging at the cellular level in tissue. Recently, it has been determined that reflectance confocal microscopy (RCM) is capable of imaging eosinophils within the esophagus. One exemplary embodiment of RCM is termed spectrally encoded confocal microscopy (SECM), which has the advantages that it can be miniaturized and incorporated into a small-diameter, flexible probe and can obtain images at very high speeds (e.g., 10-100× faster than video rate), a feature that enables microscopic screening of entire luminal organs in realistic procedural times.

One objective of this invention is to provide an accurate and inexpensive diagnostic tool for EoE. It is possible to accomplish this goal utilizing RCM and SECM technology so that it can be incorporated into a small, flexible transnasal or transoral probe capable of automatically scanning the esophagus to identify eosinophils. This device can be utilized in the outpatient setting without requiring sedation, endoscopy, or biopsies, thereby decreasing the inconvenience and cost of monitoring eosinophil counts in patients undergoing therapy for EoE.

### Eosinophilic Esophagitis

Eosinophilic esophagitis (EoE or EE) is an inflammatory disease of the esophagus that is primarily caused by food allergy. Approximately 50-70% of patients with EoE have another concomitant atopic disease. EoE affects both children and adults, is more common in Caucasians (>90%) and in males (2-4:1 ratio). The distinguishing feature of EoE is eosinophil infiltration within the esophageal squamous epithelium that does not resolve following acid reduction therapy and is not present elsewhere throughout the GI tract. Patients with EoE can experience a variety of unpleasant symptoms such as chest pain, nausea, vomiting, dysphagia, food impaction, and failure to thrive. While the natural history of this disease is not fully understood; experts fear that longstanding eosinophilic inflammation may give rise to permanent esophageal damage, leading to fibrosis of the lamina propria, esophageal narrowing, stricture, and perforation.

The significance of EoE was underappreciated for twenty years. Over the past decade, however, pioneering work by a number of investigators has brought the importance of this disease to light. During this period, the number of patients found to have EoE has risen dramatically. Emerging data suggests that the prevalence of EoE may approach 0.01-0.1% of the general population, possibly higher than that of other inflammatory bowel diseases such as Crohn's. The incidence of EoE is also rapidly increasing, mirroring the increase in atopic disease incidence found in Western countries over the same time period. Recognition of EoE is now much greater than before; this, combined with its high prevalence and incidence has pediatric and adult gastroenterologists struggling to cope with an influx of patients presenting with this disease.

### EoE Diagnosis

Recently, diagnosis of EoE can require an upper endoscopy with biopsy. At upper endoscopy, EoE patients can have findings of diminished vascularity, longitudinal furrowing, friability, microabscesses (white specks), exudates, esophageal rings (trachealization of the esophagus), narrow-caliber esophagus, strictures, and food impaction. The accuracy of endoscopy alone for EoE is modest, however, as these findings may be seen in other diseases and the esophagus may be endoscopically normal in up to 30% of cases. The only objective method for EoE diagnosis is endoscopic biopsy. The current standard of care is pinch biopsies obtained at the distal (˜5 cm from the gastroesophageal junction (GEJ)) and proximal (˜15 cm from the GEJ) portions of the esophagus. Biopsies are taken from sites with abnormal endoscopic findings and also from locations that do not show visual evidence of disease. The number of biopsies that are obtained is also important as a recent study showed that the histopathologic diagnosis of EoE was only correctly rendered in 55% of patients with one biopsy, but increased to 100% after 5 biopsies.¹ Finding greater than 15 eosinophils per high power field (HPF=400×) in any one of the biopsy specimens renders a diagnosis of EoE. Other histopathologic findings, such as eosinophil degranulation, microabscesses, superficial layering of the eosinophils, basal layer hyperplasia, and increased numbers of mast cells and other leukocytes are also seen in EoE, but are currently not required for diagnosis.

Primary EoE diagnosis can require pathologic findings of elevated esophageal eosinophils in the absence of acid reflux and without the involvement of other segments of the GI tract. As a result, at the time of first diagnosis, biopsies are also acquired from the stomach and duodenum.³ In addition, for primary diagnosis, the biopsies are usually repeated following one month of proton pump inhibitors (PPI) to rule out GERD etiology. Follow up diagnosis by endoscopic biopsy is much more common and utilized to manage multiple therapy decisions and regimens throughout the course of the disease.

### EoE Treatment

Both pharmacologic and dietary therapy can relieve symptoms and return the esophagus to histologic normalcy. The only established pharmacologic therapy is corticosteroids. Systemic steroids are recommended for urgent relief of acute symptoms and oral or swallowed topical routes of administration for maintenance. The primary complication of oral and topical corticosteroids is esophageal candidiasis, which has been observed in up to 20% of patients. The main limitation of corticosteroids is that EoE universally recurs when they are withdrawn, and as a result, if corticosteroids are the only line of therapy, they must be continued throughout the duration of the disease. Follow up endoscopic intervals with biopsy to determine when the steroid dosages must be adjusted or discontinued are currently under debate.⁴ The long term effects of corticosteroids for maintenance of remission in EoE patients are also not well understood.⁴ Other newer agents, such as anti-IL-5 and IL-13, which are antibodies to cytokines that regulate eosinophil chemotaxis, are currently under investigation.

Another effective therapy option for EoE patients is strict dietary elimination to remove the offending antigen. The most effective diet is the elemental diet, which provides nutrients as small molecular weight compounds, including proteins as amino acids or peptides, carbohydrates as oligosaccharides or monosaccharides, and fats as medium-chain triglycerides. In pediatric studies, administration of an elemental diet has been shown to resolve EoE in up to 98% of cases. However, the elemental diet is very restrictive, poorly tolerated, and may have to be administered by a feeding tube. As a result, less stringent elimination diets have been proposed, such as the six-food elimination diet (milk, egg, soy, wheat, shellfish, and nuts), which in one study effectively treated EoE in approximately 75% of patients. Methods for finding the culprit dietary antigen, such as skin prick and allergy patch testing (APT) have been proposed, but are not yet accurate enough to be recommended for managing the diets of EoE patients. Dietary elimination is very difficult for patients to tolerate and as a result, foods are gradually reintroduced into the diet until symptoms and/or esophageal eosinophils recur.

### Improvement of Monitoring of Eosinophils in EoE Patients

Because of the impact of EoE on quality of life and the concern that this disease may progress to esophageal fibrosis with unknown long-term risks, experts recommend that EoE be treated until symptoms and the eosinophilic infiltrate are resolved. Management of these patients requires many repeat endoscopic biopsies during the food reintroduction and/or corticosteroid dosing process to assess esophageal eosinophils. This process is inconvenient and taxing to patients and their families. It is also associated with a small but real risk of complications associated with endoscopy. In addition, at an average of currently about $1000 per procedure, endoscopic biopsy for EoE monitoring can be a significant expense to our health care system. These considerations have led to a preference to determine less invasive and more cost effective ways to obtain esophageal eosinophil counts.

### Cost of Sedation and Upper Endoscopy

Sedation, via IV administration of sedatives and narcotic analgesics, is the single most important contributor to the high cost of upper endoscopy and is estimated to account for 30-50% of the total procedural cost. Because of the mortality and morbidity associated with complications related to sedation, patients must undergo continuous cardiopulmonary monitoring and nursing support during the endoscopic procedure. Post-procedural recovery also contributes to the expense, as it requires additional nursing, monitoring, and patient care in a large and specialized physical space. After discharge, patients frequently need to be escorted home, and adults lose at least a day's work. Finally patient tolerance for sedated procedures is lower than for unsedated procedures. In addition to the expense of sedation, other modifiable costs of upper endoscopy include physician time and biopsy processing; assessing esophageal eosinophils would undoubtedly be less expensive if a nurse or technician could conduct the procedure. Biopsies can add to the cost at about $150 per specimen. Taking these factors into account, up to half of the total cost of upper endoscopy can be a result of sedation.

### Potential Blood Biomarkers for EoE

Blood biomarkers that may be surrogates for esophageal eosinophils are actively being investigated to overcome the high cost and inconvenience limitations of endoscopic biopsy. Research into the pathophysiology of this disease has indicated that EoE results from a TH2 inflammatory process, mediated by IL-5 and IL-13 and dependent on Eotaxin-3. In addition to these cytokines, peripheral blood eosinophils, serum IgE, CD23, and Eosinophil Derived Neurotoxin have also been proposed as potential biomarker surrogates for biopsy eosinophil levels. While some correlations between these biomarkers and EoE have been found, research in this area is early and important parameters such as the cost of these tests, robust cutoff values, and effects of age, symptoms, and gender have yet to be determined. It may be preferable to determine whether the specificity of these biomarkers for esophageal eosinophils, especially in patients who are likely to have other coexistent atopic diseases, is sufficient for guiding therapy decisions.

Unsedated transnasal procedures.

For example, transnasal and transoral access to the esophagus can be attained in the outpatient setting without sedation for both adult and pediatric patient populations. When passing a catheter in the unsedated patient, transnasal access is generally better tolerated than the transoral approach because of a more vigorous gag reflex encountered in transoral procedures. Standard transnasal procedures, such as nasogastric tube (NG tube) insertion, are conducted in millions of patients annually with few if any major complications. With NG tube insertion, a flexible, 10-18F (3-6 mm) diameter tube is first measured from the nares to the xiphoid process. It is then lubricated with a anesthetic gel and inserted into one of the patient's nares that has been previously anesthetized with a topical lidocaine spray. The tube is aimed down and back and advanced; as the tube passes the posterior pharynx, the patient is asked to swallow or sip water through a straw to suppress the gag reflex. When the tube passes the pharynx, it is easily advanced through the esophagus to the stomach. Confirmation that the tube is in the stomach can be accomplished by injecting air through the tube and auscultating with a stethoscope or by measuring the pH of aspirated fluid. The tube can then be retracted into the esophagus. Following the procedure, patients can usually resume regular activity immediately.

### Reflectance Confocal Microscopy

Reflectance confocal microscopy (RCM), can be suited for imaging esophageal eosinophils in EoE patients, as it is a non-invasive optical imaging method that enables the visualization of cellular structures at approximately 1 μm resolution, and does not require the administration of exogenous contrast agents. RCM rejects or ignores multiply scattered light from tissue and detects the singly backscattered photons that contain structural information by employing confocal selection of light reflected from a tightly focused beam. Most commonly, RCM is implemented by rapidly scanning a focused beam in a plane parallel to the tissue surface, resulting in transverse or en face images of tissue. The large numerical aperture (NA>0.3) of the objective lens used in RCM yields a very high spatial resolution. The imaging depth of penetration of RCM (several hundred micrometers) is well suited to the detection of EoE, which typically manifests near the luminal surface.

### Possible Limitations of Conventional Confocal Microscopy for Clinical Application

All internal organ confocal microscopy imaging modalities proposed to date provide microscopic images under endoscopic guidance and only at discrete locations—the so-called “point sampling” method. Point sampling can be inherent to RCM since it has an extremely limited field of view (in the range of about 200-500 μm), which is less than that of a pinch biopsy used for EoE diagnosis. With the transnasal approach proposed here, the diagnostic probe can be inserted without endoscopic guidance and the data acquired automatically. As a result, relatively large areas of the esophagus should be imaged in order to be certain that this “unguided” method captures information from areas that contain elevated eosinophils and that are representative of the overall disease state.

### Comprehensive Volumetric Microscopy

This mode of operation may need a paradigm shift, which can be termed “Comprehensive Volumetric Microscopy (CVM),” or the capability to obtain microscopic images of the epithelium of large mucosal areas in three-dimensions. Others in the field are now recognizing the value of this concept; interesting mosaicing approaches have been devised to stitch multiple confocal images together, covering a few mm², but this technology is still far from the comprehensive imaging that is required to scan a sufficient (10+ cm) length of the esophagus for eosinophils.

For CVM, imaging speeds should be increased by at least an order of magnitude above video rate, due to the tremendous bandwidth of the microscopic information and the constraint of obtaining this data in a realistic procedural time (<10 min). In addition, a RCM probe must be developed to automatically scan the microscope over these large tissue surface areas rapidly and with a high degree of precision. Recently, CVM can be implemented using a second-generation form of OCT, called optical frequency domain imaging (OFDI), and rapid helically scanning catheters. This research has enabled the acquisition of three-dimensional microscopic images of the entire distal esophagus (6.0 cm length) in a few minutes and long (˜10 cm) segments of coronary arteries in patients in less than 5 seconds. While OFDI systems and method shows can be very helpful for various clinical applications, the use of approximately 10 μm resolution by such exemplary systems and methods may not be sufficient for identifying individual cells.

### Reflectance Confocal Microscopy of Eosinophils

Exemplary embodiments of RCM apparatus and methods of esophageal eosinophils has not been previously described. Thus, according to the exemplary embodiments of the present disclosure, as shown in FIG. 1, such cells 100 can be visualized by RCM with a high degree of contrast. For example, the physical basis for this capability is likely primarily related to the large size, high refractive index, and abundance of the granules within the eosinophil's cytoplasm. Modeling of light scattering from cells can indicate that organelles that are large and that have a high refractive index gradient exhibit stronger optical backscattering and therefore a stronger RCM signal. Of the three major types of granules present in the cytoplasm of eosinophils, ‘specific’ crystalloid granules, which contain major basic protein, eosinophil peroxidase, eosinophil cationic protein, and eosinophil derived neurotoxin, can be the most abundant. These crystalloid granules can measure between about 0.5-1.3 μm in diameter, significantly larger than those found in other granulocytes. While the refractive index of these granules has not been directly measured; it can be high, e.g., approaching 1.6, due to the density and types of crystalloid proteins contained within. Since the refractive index of the cytoplasm ranges from 1.35-1.37, the refractive index gradient induced by these granules is very large. In contrast, squamous cells do not have such granules and have a relatively homogeneous cytoplasm. As a result, the eosinophil's cytoplasm can backscatter light more intensely than the surrounding esophageal epithelium, resulting in high RCM image contrast. In addition to granules, nuclei can also generate a RCM signal due to their size and the refractive index of chromatin (1.39-1.45). Eosinophils can therefore be further discriminated in RCM images by utilizing nuclear morphology cues, such their bi-lobed shape.

### Confocal Microscopy for Internal Organ Systems

While RCM apparatus and methods has been described as it is applied in the skin, small confocal microscopy probes for internal organ imaging were not developed to technical challenges associated with miniaturizing a scanning microscope. One difficulty can be the development of a mechanism for rapidly raster-scanning the focused beam at the distal end of a small-diameter, flexible probe. A variety of approaches have been attempted to address this problem, including the use of distal micro electro mechanical systems (MEMS) beam scanning devices, and proximal scanning of single-mode fiber bundles. Another challenge can be the miniaturization of high NA objectives used for optical sectioning.

This exemplary need has led to a development of an exemplary embodiment of an apparatus and a method of RCM according to the present disclosure, which can be termed spectrally encoded confocal microscopy (SECM) that can be configured to rapidly obtain higher-resolution CVM images via, e.g., a small-diameter probe.

### OBJECTS AND SUMMARY OF EXEMPLARY EMBODIMENTS OF DISCLOSURE

According to one exemplary embodiment of the present disclosure, exemplary RCM techniques, methods and apparatus according to the present disclosure can be configured to identify esophageal eosinophils.

One of the objects of exemplary embodiments of the present disclosure is to determine the presence or absence of esophageal eosinophils in human patients. Another object of the present disclosure is to count, in a manual, semi-automatic, or computer processing manner, the number of eosinophils in human patients. A further object of the present disclosure is to obtain cross-sectional images of tissue at the cellular level to enable the counting of eosinophils. Another object of the exemplary embodiments of the present disclosure present disclosure is to identify other cells, such as leukocytes, including mast cells, lymphocytes, basophils, and neutrophils, that may also be elevated in EoE. A further object of the exemplary embodiments of the present disclosure present disclosure is to provide a device for evaluating other histologic features of EoE in vivo, including basal layer hyperplasia, abscess, eosinophil degranulation, and lamina propria fibrosis.

Still another object of the present disclosure is to provide an exemplary embodiment of an RCM or SECM apparatus and transnasal probe for imaging esophageal eosinophils.

For example, a transnasal or transoral RCM probe can be designed and fabricated based on the nasogastric (NG) tube, e.g., a predicate device. A pressure sensor can be utilized to facilitate placement of the RCM probe's optics near the gastroesophageal junction. The probe's optics can scan from the distal to proximal esophagus, creating a three-dimensional RCM image that covers a surface area of a surface area of the esophagus that may range from 0-1000 mm². In addition, a faster and higher resolution RCM system can be provided to facilitate the scan to be completed in a few minutes.

Thus, in accordance with the exemplary embodiments of the present disclosure, it is possible to provide devices and methods which can measure esophageal eosinophils directly, and without the cost and complexities of endoscopic biopsy. The exemplary embodiments can use reflectance confocal microscopy techniques, e.g., implemented through a flexible, small-diameter (approximately 3 mm) transnasal probe. The exemplary device can be configured or structured to be deployed without sedation in the outpatient setting and operated by a technician. Once inserted, images from the entire length of the esophagus can be automatically obtained and analyzed by a computer to provide eosinophil counts. The exemplary embodiments of the present disclosure can provide a cost-effective and less invasive tissue image-based biomarker for EoE that can be used to follow these patients during their therapeutic course. Additionally, the exemplary device can be utilized to study this disease to answer questions about the pathophysiology and natural history of EoE.

The exemplary embodiments of the present disclosure can be utilized, e.g., in research and clinical applications for other diseases associated with elevated tissue eosinophils. For instance, certain subtypes of asthma are characterized by the presence of airway eosinophilia and the technology developed here may provide a new means for personalizing and monitoring the response to asthma therapy. Improving understanding, diagnosis, and therapeutic monitoring of other eosinophilic diseases such as eosinophilic gastritis, gastroenteritis, and colitis, as well as hypereosinophilic syndromes, would also be facilitated by the exemplary embodiments of the present disclosure for visualizing these cells in vivo.

Figure According to further exemplary embodiments of the present disclosure, the transnasal SECM systems, apparatus and methods cancan facilitate a clinical use thereof, and detect esophageal eosinophils over large surface areas. This exemplary capability can greatly improve the management of EoE patients, in accordance with current guidelines. Given the relative newness of this disease entity, however, today's guidelines are primarily based on Class 3 evidence such as case reports and individual clinical experiences. Because of the ability of the exemplary SECM systems, apparatus and methods can according to the present disclosure to longitudinally monitor esophageal eosinophils in a minimally invasive manner, they can be used to determine the role of eosinophils in precipitating symptoms and long-term complications.

Thus, an exemplary embodiment of apparatus and method according to the present disclosure can be provided. For example, using at least one first arrangement, it is possible to direct at least one first electro-magnetic radiation to at least one portion of tissue within a body. Using at least one second arrangement, it is possible to receive at least one second electro-magnetic radiation provided from the portion, which is based on the first electro-magnetic radiation. Further, using at least one third arrangement, it is possible to differentiate at least one particular cell which is eosinophil, mast cell, basophil, monocyte and/or nutrophil from other cells in the portion based on the second electro-magnetic radiation.

According to one exemplary embodiment of the present disclosure, the second electro-magnetic radiation can be reflected from the portion(s). The third arrangement can be configured to image the particular cell(s). The third arrangement can be configured to image the particular cell(s) over a region of the tissue that greater than an are of 1 mm². The third arrangement can also be configured to image the particular cell(s) in three-dimensions, and/or to image a cross-section of the particular cell(s). The tissue can be a luminal organ, and the luminal organ can be an esophagus and/or a pulmonary airway.

According to another exemplary embodiment of the present disclosure, the first arrangement and/or the second arrangement can be provided in a catheter. The catheter can be structured and/or sized to be inserted to reach the tissue transorally or transnasally, and/or can have a cross-sectional diameter of less than 5 mm. The catheter can include a balloon arrangement, and the balloon arrangement can contain an auto-focusing arrangement which is configured to auto-focus on the portion. The catheter can be facilitated in a nasogastric tube, and/or can include a wound cable. In addition or as an alternative, the catheter can include a further arrangement which is configured to measure pressure of the tissue within a body. The catheter can have a portion to be inserted into the body which is substantially flexible.

In yet another exemplary embodiment of the present disclosure, the particular cell(s) can include a plurality of particular cells, and the arrangement can be further configured to determine a number of the particular cells, and/or automatically count the number of the particular cells. Further, the second arrangement can be configured to receive a confocal light and/or a spectrally encoded confocal light. In addition, the second arrangement can be configured to receive and detect a florescent electro-magnetic radiation.

According to a further exemplary embodiment of the present disclosure, the first arrangement and/or the second arrangements can include a further arrangement which is configured to spectrally disperse the first electro-magnetic radiation and/or the second electro-magnetic radiation, respectively. The first arrangement and/or the arrangement can contain at least one optical fiber arrangement which has multiple wave-guiding regions. The optical fiber arrangement can include a double-clad fiber core. Further, the first arrangement can be configured to transmit a broadband light and/or light whose frequency changes over time. Further, the third arrangement can be additionally configured to differentiate basal layer hyperplasia, abscess, eosinophil degranulation and/or lamina propria fibrosis from other cells. The particular cell(s) to be differentiated can be or include eosinophil. In addition, the third arrangement can differentiate the particular cell(s) based on (a) a strength of a signal from a cytoplasm and/or (ii) a shape of a nucleus of the particular cell.

These and other objects, features and advantages of the exemplary embodiment of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of exemplary embodiments of the present disclosure can become apparent from the following detailed description taken in conjunction with the accompanying figures showing illustrative embodiments of the present disclosure, in which:

FIG. 1 is an exemplary SECM image of a biopsy sample from a patient with EoE, demonstrating numerous white dots that correspond to esophageal eosinophils;

FIG. 2 is a schematic diagram of an exemplary embodiment of an SECM system and probe in accordance with the present disclosure;

FIG. 3 is an exemplary SECM image of a human esophageal biopsy sample, illustrating the gastroesophageal junction, squamous epithelium, and gastric cardia.1;

FIG. 4 is an exemplary SECM image of a human esophageal biopsy sample, demonstrating squamous epithelium, keratinocyte cell walls, and the lamina propria papillae;

FIG. 5 are exemplary SECM and histology images of an esophageal biopsy obtained from a 31-year-old male with a history of EoE;

FIG. 6 are exemplary SECM and histology images of an esophageal biopsy from a patient with a history of EoE;

FIGS. 7A-F are exemplary images from a 29-year-old male with dysphagia;

FIG. 8A is a schematic diagram of an esophageal optical imaging catheter;

FIG. 8B is an exemplary photograph of a catheter for esophageal imaging;

FIG. 8C is a schematic diagram of an exemplary esophageal balloon catheter;

FIG. 8D is an exemplary photograph of a balloon catheter for esophageal imaging;

FIGS. 9A-9C are exemplary images from a patient with Barrett's esophagus obtained in vivo;

FIGS. 10A and 10B are exemplary images from a patient with Barrett's esophagus and biopsy-proven dysplasia obtained in vivo;

FIG. 11A is a schematic diagram of an exemplary SECM bench top probe in accordance with the present disclosure which includes 50/50 beam splitter;

FIG. 11B is an exemplary photograph of an imaging arm;

FIG. 11C is an exemplary photograph of an exemplary motor-mounted housing;

FIGS. 12A-12E are exemplary comprehensive SECM images from a lens paper phantom, displayed at increasing magnifications;

FIG. 13 is an exemplary image/data of Automated SECM eosinophil counting based on bimodal histogram segmentation and size thresholding;

FIG. 14A-C are exemplary SECM cross-sectional imaged obtained from biopsies from patients with a low (A), medium (B), and high (C) number of eosinophils.

FIG. 14D is an exemplary scatter plot illustrating SECM eosinophil counts versus histology eosinophil counts, demonstrating a high degree of correlation between the two measurements;

FIG. 15 is a schematic diagram of an exemplary embodiment of a transnasal SECM device according to the present disclosure;

FIG. 16 is a schematic diagram of an exemplary embodiment of a fiber-optic Fabry-Perot sensor for pressure measurements according to the present disclosure;

FIG. 17 is a schematic diagram of an exemplary embodiment of the SECM probe according to the present disclosure;

FIG. 18 is a schematic diagram of an exemplary embodiment of an SECM system console according to the present disclosure;

FIG. 19 is a schematic diagram of an exemplary embodiment of a rotary junction with a single-mode/multi-mode splitter and a double-clad fiber according to the present disclosure;

FIG. 20 is a graph of an exemplary SECM sensitivity noise analysis plot according to the present disclosure;

FIGS. 21A and 21B are exemplary images and graphs of ZEMAX modeling of exemplary probe optics;

FIGS. 22A and 22B are exemplary diagrams of three-dimensional modeling of the exemplary probe assembly according to the present disclosure;

FIGS. 23A and 23B are schematic diagrams of a transnasal balloon-centering SECM probe according to the exemplary embodiment of the present disclosure;

FIG. 24 is a schematic diagram of an exemplary balloon-centering SECM probe according to the present disclosure;

FIG. 25 is a schematic diagram of an exemplary spectrally-encoded illumination on tissue with feedback according to the present disclosure;

FIGS. 26A and 26B are exemplary SECM image and intensity profile, respectively, used for adaptive focusing;

FIG. 27 is an exemplary photograph of a SECM probe with an autofocusing capability according to the present disclosure;

FIGS. 28A-28D are exemplary images of a lens paper phantom obtained with the exemplary probe of FIG. 27, whereas FIG. 28A is an exemplary cylindrical presentation of image obtained without adaptive focusing, FIG. 28B is an exemplary magnified view red dotted region in FIG. 28A showing areas of SECM signal dropout, FIG. 28C is an exemplary cylindrical presentation of image obtained with adaptive focusing, and FIG. 28D is an exemplary magnified view of red dotted region in FIG. 28C, demonstrating complete imaging of the phantom;

FIGS. 29A-29C are exemplary illustration of a normal esophagus demonstrating a basal cell layer of 20 μm thickness, whereas FIG. 29A is an exemplary histopathologic section (e.g., H&E stain; original magnification 20×) showing normal squamous mucosa with no evidence of eosinophils, FIG. 29B is the corresponding SECM image of normal squamous mucosa showing transversely sectioned papillae at low magnification, FIG. 29C is an exemplary high magnification view of the SECM image demonstrating regions of higher reflectance surrounding the papillae;

FIGS. 30A-30D are exemplary illustrations obtained from an 11 y/o male patient with EoE, whereas FIG. 30A is an exemplary videoendoscopy image demonstrating faint evidence of rings and a slightly diminished vascular pattern, FIG. 30B is a histopathology (e.g., H&E stain) demonstrating an abundance of eosinophils within the squamous epithelium (e.g., greater than about 25 eos/HPF), FIG. 30C is an exemplary SECM image at low magnification demonstrating a diffuse infiltration of highly scattering cells, and FIG. 30D is an exemplary higher magnification view of the yellow dotted region in FIG. 30C showing that these cells are eosinophils with bi-lobed nuclei (inset);

FIGS. 31A-31F are exemplary illustrations of SECM and histopathology acquired from a 6-year-old male patient with EoE, demonstrating one-to-one cellular level matches, whereas FIG. 31A is an exemplary digital histology (H&E stain; original magnification 20×) showing the papillae structure and intraepithelial eosinophils, FIG. 31B is an exemplary expanded view of FIG. 31A showing intact eosinophils as well as eosinophilic cytoplasm fragments, FIGS. 31C and 31D are exemplary color transformation (R/G) of histology in FIG. 31A and FIG. 31B, respectively, allowing the eosinophils to be seen more clearly, and FIGS. 31E and 31F are exemplary corresponding registered SECM images demonstrating the papillae and highly reflecting cells that are directly matched to the eosinophils seen by histology with scale bars=about 100 μm; and

FIGS. 32A-32F exemplary illustrations of histology and SECM showing microscopic features of EoE, whereas FIG. 32A is an exemplary histology image of eosinophilic abscess showing aggregates of eosinophils in the esophageal epithelium, FIG. 32B is the exemplary corresponding SECM image demonstrating a large number of closely spaced eosinophils, FIG. 32C is a histology image of eosinophil degranulation showing stellate eosinophils and extracellular eosinophilic granules, FIG. 32D is the exemplary corresponding SECM image demonstrating cells that have an irregular shape and poorly delineated cell boundaries with the extracellular highly scattering granular densities consistent with granules seen on the histology, FIG. 32E is an exemplary histology image of basal cell hyperplasia showing a thickened basal layer and elongated papillae, and FIG. 32F is the exemplary corresponding SECM image demonstrating numerous eosinophils and a thickened highly reflecting layer (e.g., about 70 μm) surrounding the papillae of the lamina propria (which can be compared to the layer surrounding the papillae seen in the normal esophagus of FIG. 29C), Scale bars=100 μm,

Throughout the figures, the same reference numerals and characters, unless otherwise stated, are used to denote like features, elements, components or portions of the illustrated embodiments. Moreover, while the subject disclosure will now be described in detail with reference to the figures, it is done so in connection with the illustrative embodiments. It is intended that changes and modifications can be made to the described embodiments without departing from the true scope and spirit of the subject disclosure.

### DETAILED DESCRIPTION OF EXEMPLARY EMBODIMENTS OF DISCLOSURE

### Preliminary Information

Described herein is a feasibility of a transnasal SECM device for eosinophil counting, including results from a esophageal biopsy study, descriptions of OFDI clinical esophageal imaging devices, and a bench top SECM probe prototype that incorporates the key components required for the implementation of a transnasal SECM probe.

### Spectrally Encoded Confocal Microscopy

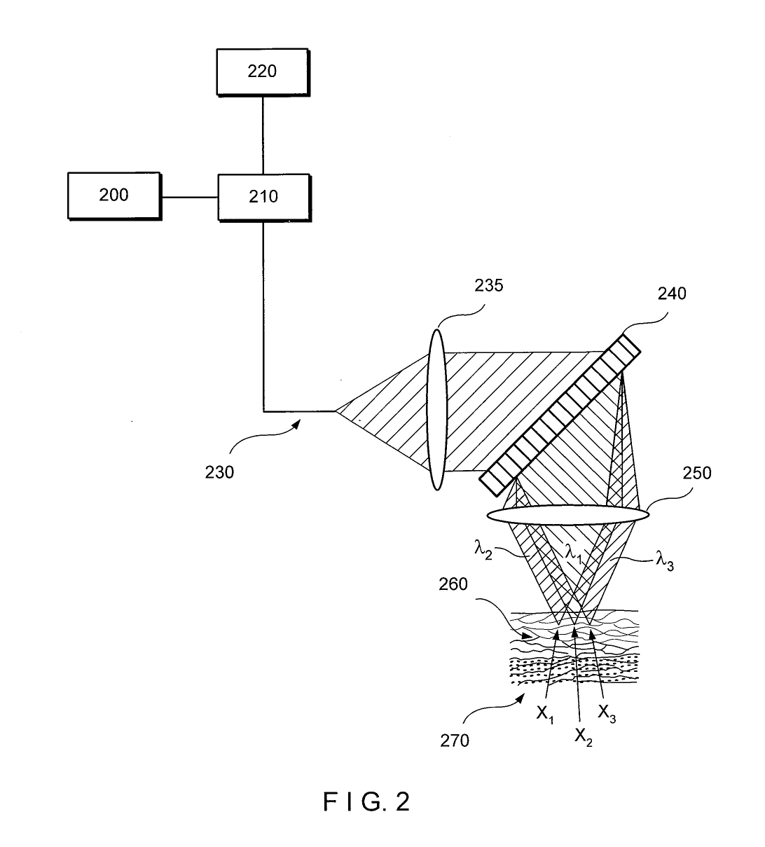

FIG. 2 shows an exemplary embodiment of a SECM system/apparatus which utilizes a single fiber-optic confocal microscopy arrangement for imaging. In particular, the exemplary system/apparatus shown in FIG. 2 includes a broad bandwidth or a wavelength-swept light source 200 that is input into a coupler or circulator 210 to encode one dimension of spatial information in the optical spectrum. At the distal end of a probe of the exemplary system/apparatus, the output from a core of a single-mode or dual-clad fiber 230 illuminates a transmission diffraction grating 240. An objective lens 250 can focus each diffracted wavelength of one or more electro-magnetic radiations (e.g., light) to a distinct spatial location 260 within a specimen 270, tissue or another anatomical structure, or plurality thereof. After reflecting from the tissue, the electro-magnetic radiation(s) pass back through the lens 250, such electro-magnetic radiations are recombined by the grating 240, and collected by the fiber 230. The aperture of the fiber can provides a spatial filtering arrangement/structure to reject or filter out an out-of-focus electro-magnetic radiation.

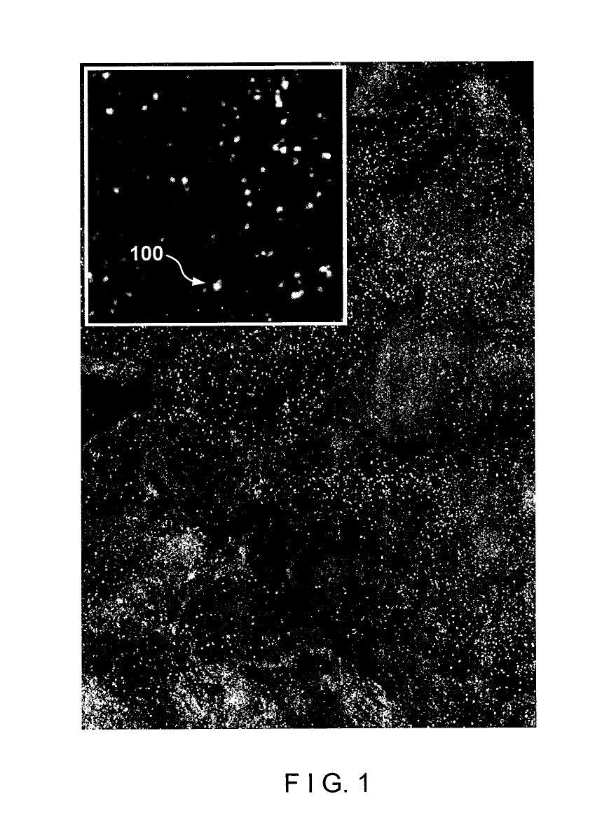

Externally from the probe (e.g., within the console of the exemplary system/apparatus), the spectrum of the returned electro-magnetic radiation can measured and/or converted into a confocal reflectance as a function of transverse displacement within the sample using a detector or a spectrometer or other spectral detecting arrangement/system 220 which is/are known to those having ordinary skill in the art. Spectral decoding of a line in the image can be performed very rapidly, e.g., at rates of up to about 400 kHz, which can be approximately 25 times that of video rate confocal microscopy systems, and over about 250 times faster than some endoscopic RCM systems. The other transverse axes of the image can be obtained by relatively slow and straightforward mechanical actuation, such as helical scanning, that can be used for a wide variety of endoscopic probes. Similarly to other RCM techniques, images obtained by exemplary SECM systems, apparatus and methods can facilitate a visualization of subcellular-level microstructure, as shown in FIG. 1, that facilitates the identification of eosinophils. Exemplary SECM systems, apparatus and methods can according to the present disclosure can be provided so that they may be utilized for GI screening and have demonstrated a bench top exemplary SECM probe of scanning an area equivalent to that of the distal esophagus (e.g., about 5 cm length, and about 2.5 cm diameter), at 15 distinct depth locations, in several minutes. According to an exemplary embodiment of the present disclosure, such exemplary systems, apparatus and methods can be incorporate into or used with a transnasal probe for standalone, unsedated eosinophil counting and for making the diagnosis of other histological features associated with EoE.

### Exemplary Capabilities of SECM to Identify Eosinophils

Exemplary embodiments of systems, methods utilizing SECM techniques and/or structures according to the present disclosure can be used to diagnose Barrett's esophagus, including dysplasia and early-stage adenocarcinoma. In order to determine the accuracy of SECM for diagnosis, it is possible to SECM images of excised upper GI biopsy samples to histopathology, which also facilitates a visualization of eosinophils.

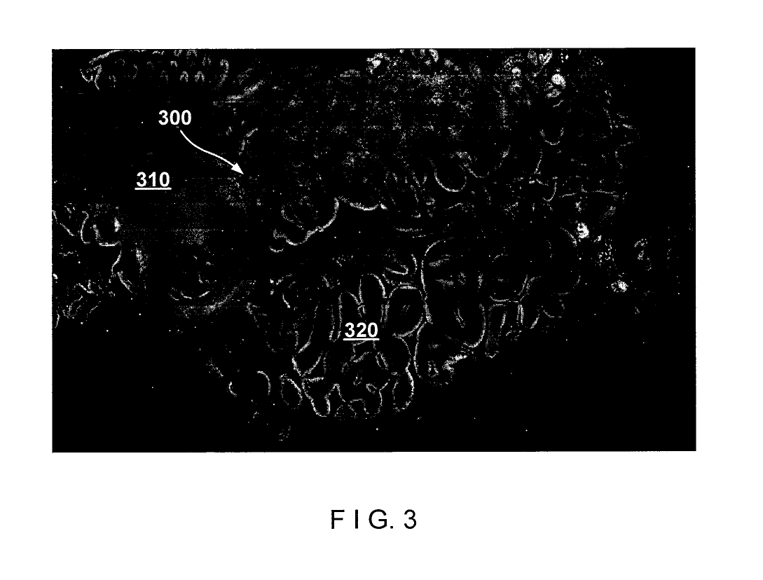

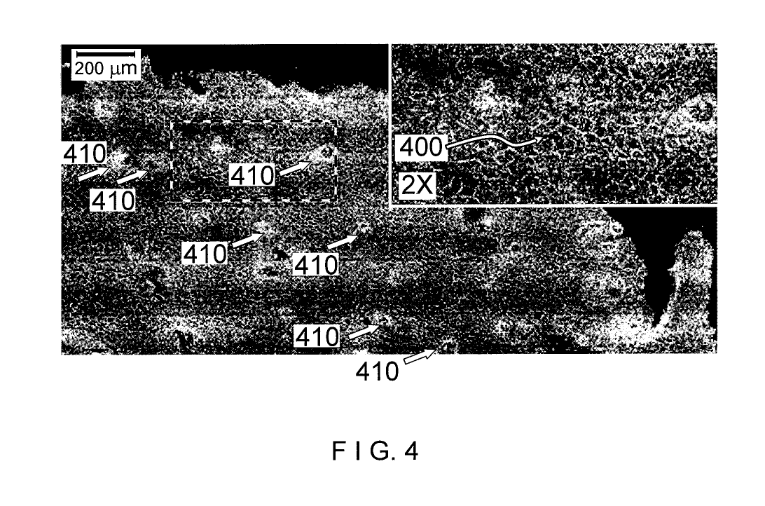

An exemplary embodiment of a SECM system according to the present disclosure can be provided that can have optical specifications for utilization of the exemplary transnasal device can used for exemplary histopathology correlation procedures. The exemplary SECM system can utilize an exemplary wavelength-swept source (central wavelength=1320 nm; bandwidth=70 nm; repetition rate=5 kHz) and an objective lens with an effective NA of 0.5.⁷³ An exemplary single-mode illumination and multi-mode detection imaging configuration can be used to reduce laser speckle noise. The transverse and axial resolutions of the exemplary SECM system can be, e.g., 2.3 μm and 9.7 μm, respectively. FIG. 3 shows an exemplary image generated using such exemplary SECM system from this biopsy analysis, demonstrating the architectural morphology of a normal gastroesophageal junction 300, including the squamous epithelium of the esophagus 310 and the gastric cardia 320. FIG. 4 shows an exemplary SECM image of normal esophageal squamous mucosa with no histologic evidence of eosinophils. Cell membranes 400 are shown in FIG. 4, as well as the papillae of the lamina propria 410.

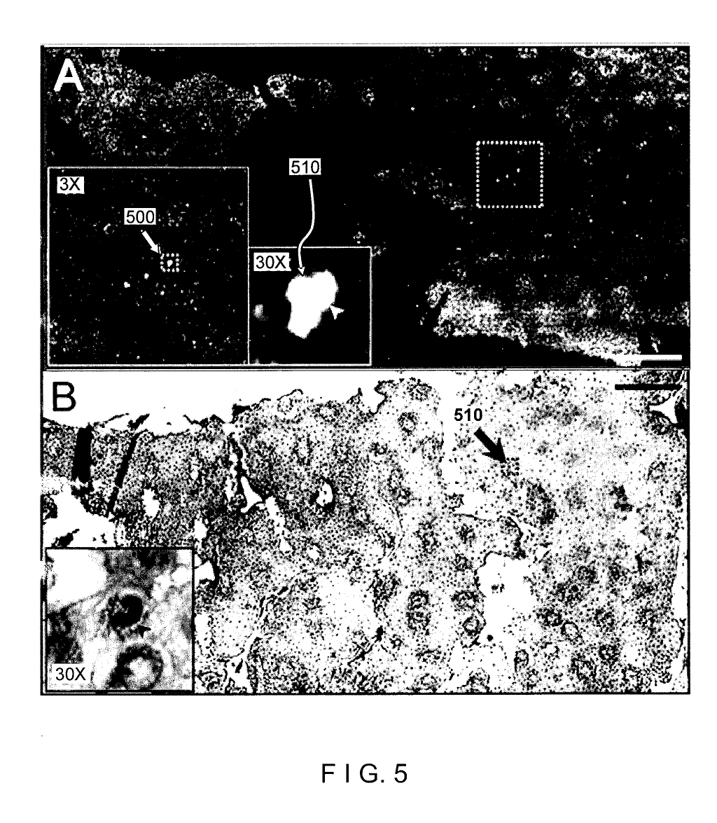

In one of the exemplary biopsies imaged in this exemplary analysis (as shown in FIGS. 5A and 5B) from a 31-year-old male with a history of EoE, the pathologic diagnosis referred to the presence of scattered esophageal eosinophils. A further inspection of the data for this case indicates that, as opposed to normal squamous epithelium (shown in FIG. 4), the exemplary SECM images of FIG. 5A illustrate punctate, highly reflecting dots 500 within the epithelium and the vessels of the lamina propria papillae. A magnified view of these exemplary regions 500 also indicates that these features are individual cells with a clearly-defined nuclei 510 and cytoplasm (as illustrated in FIG. 5A, see 30× inset thereof).

The nuclei shown in FIG. 5A has a characteristic bi-lobed appearance of eosinophils 510. The reflectance from the cytoplasm of these cells can be substantially higher than that of the surrounding squamous cells. Representative histology from this biopsy shown in FIG. 5B indicates that eosinophils 520 is present with the same or very similar spatial distribution as illustrated in the exemplary SECM image. Furthermore, the exemplary nuclear morphology 530 of these eosinophils (as shown in FIG. 5B, inset, black arrowhead) can match the exemplary nuclear morphology identified in the SECM images shown in FIG. 5A, 30× inset, white arrowhead), illustrating a bi-lobed appearance.

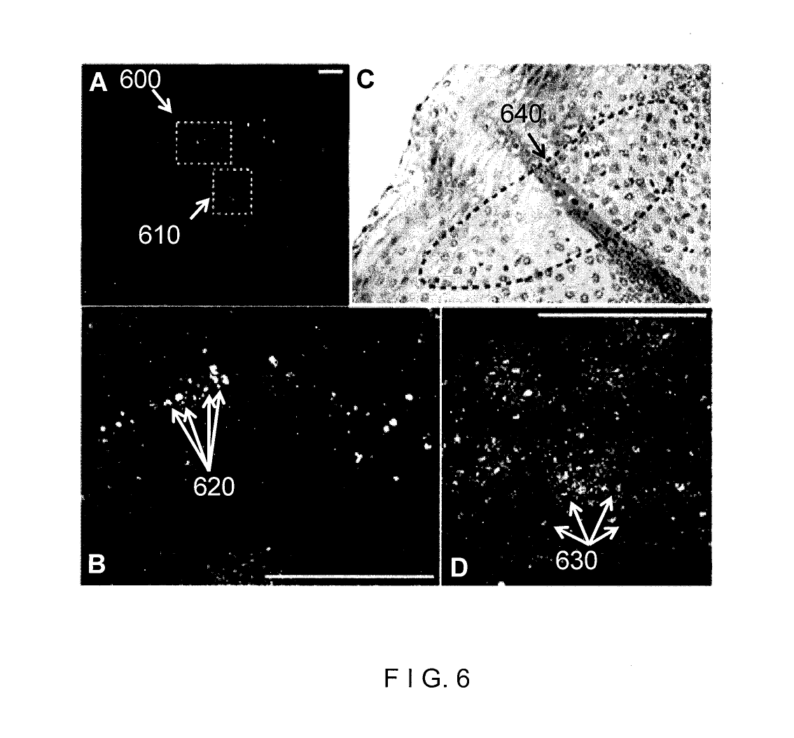

After confirming that eosinophils are visible by the exemplary SECM system, apparatus and method, a protocol can be modified to image biopsies from pediatric patients with suspicion of EoE. An exemplary pediatric biopsy can be obtained from a pediatric patient with a history of EoE. As shown in FIGS. 6A and 6B, a large cluster 600 of highly reflecting dots 620 can be seen in the exemplary SECM image acquired approximately 140 μm below the luminal surface. These cells have a scattering intensity that was at least about 10 times greater than that of the squamous cell nuclei 630 in the adjacent region 610 (as shown in FIG. 6D). Light reflected from many of the cells can saturate the detector. Thus, the exemplary diagnosis for this biopsy can be a focus of scattered eosinophils, with approximately 10 eos/HPF 640, as shown in FIG. 6C, below the histopathologic threshold of EoE.

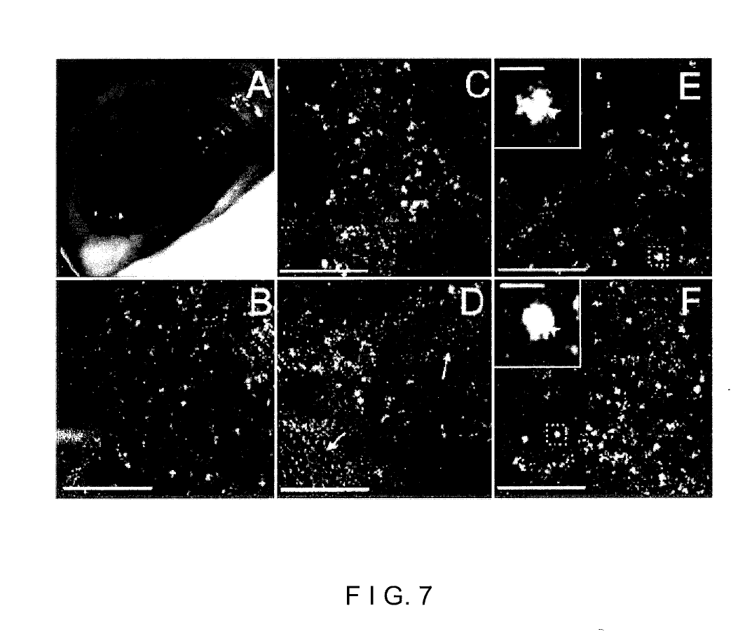

FIG. 7A depicts an exemplary video endoscopy image of another patient's esophagus, demonstrating esophageal trachealization (multiple concentric rings) that is a classic endoscopic finding of EoE. The exemplary SECM system, apparatus and method can facilitate the identification of multiple high power field regions of interest (e.g., about 250 μm×250 μm) within the squamous epithelium that showed evidence of greater than about 15 eos/HPF (as shown in FIGS. 7B-7F). An exemplary histopathologic analysis of this sample can confirm the diagnosis of EoE.

### Exemplary Comprehensive Microscopic Imaging of Esophagus

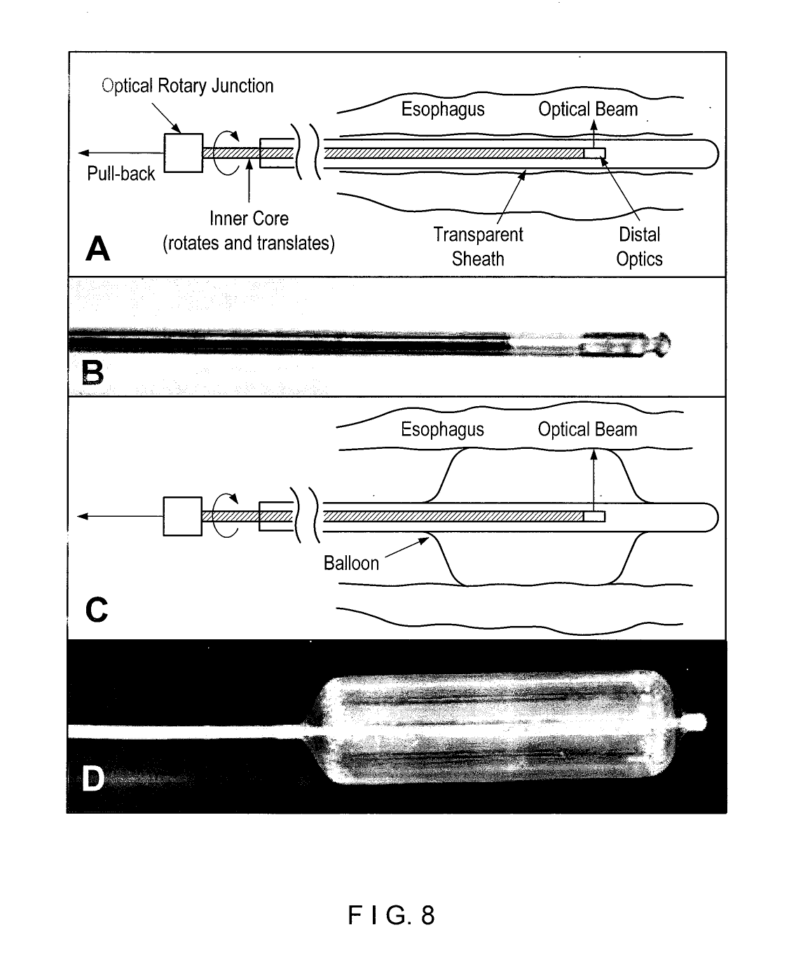

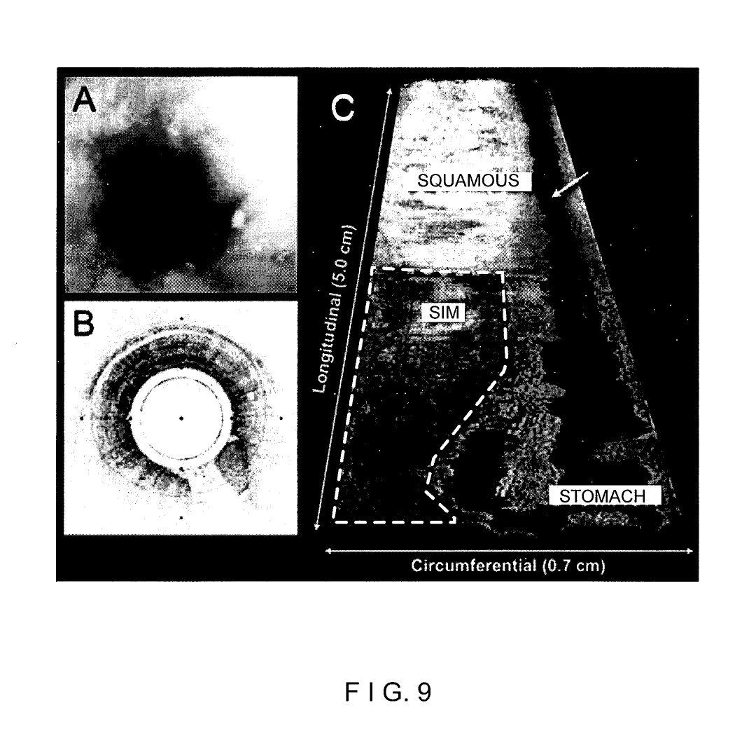

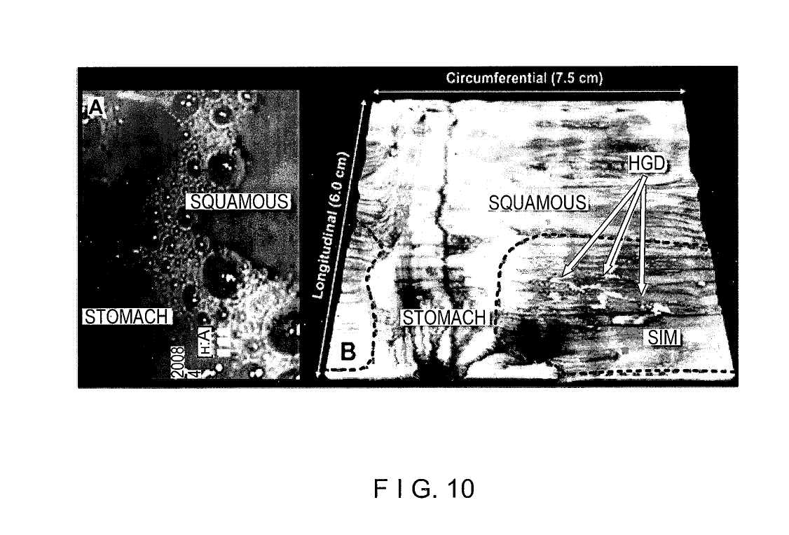

Exemplary optical coherence tomography or exemplary optical frequency domain imaging probes for imaging large regions of the esophagus can be provided. FIGS. 8A-8D illustrate certain exemplary designs and arrangements according to the present disclosure can be utilized to image large esophageal mucosal areas. One exemplary version of the exemplary catheter can contain an inner cable that can incorporate the optical fibers and distal optics (as shown in FIGS. 8A and 8B). This cable can rotate and translate within an outer transparent sheath to produce a helical scan of the esophagus. When inserted into the lumen of an uninsufflated esophagus, the esophagus can collapse around the probe, as illustrated in FIG. 8A, thus allowing, e.g., approximately 10% of the total esophageal surface area of interest to be imaged (as shown in FIG. 9C). In another exemplary embodiment of the system, apparatus and method according to the present disclosure, a centering balloon can be provided within or at the transparent sheath, as shown in FIGS. 8C and 8D). The balloon can facilitate an expansion of the esophagus, and can center the optics so that the entire circumference may be imaged (as shown in FIGS. 8C and 10B). It is also possible to modify the exemplary embodiments of the catheter arrangements described herein above so that they can be implemented transnasally and configured to perform SECM imaging to obtain cellular-resolution images of large esophageal areas.

### Exemplary Designs, Systems, Apparatus and Methods

Exemplary SECM Probe with Exemplary Components

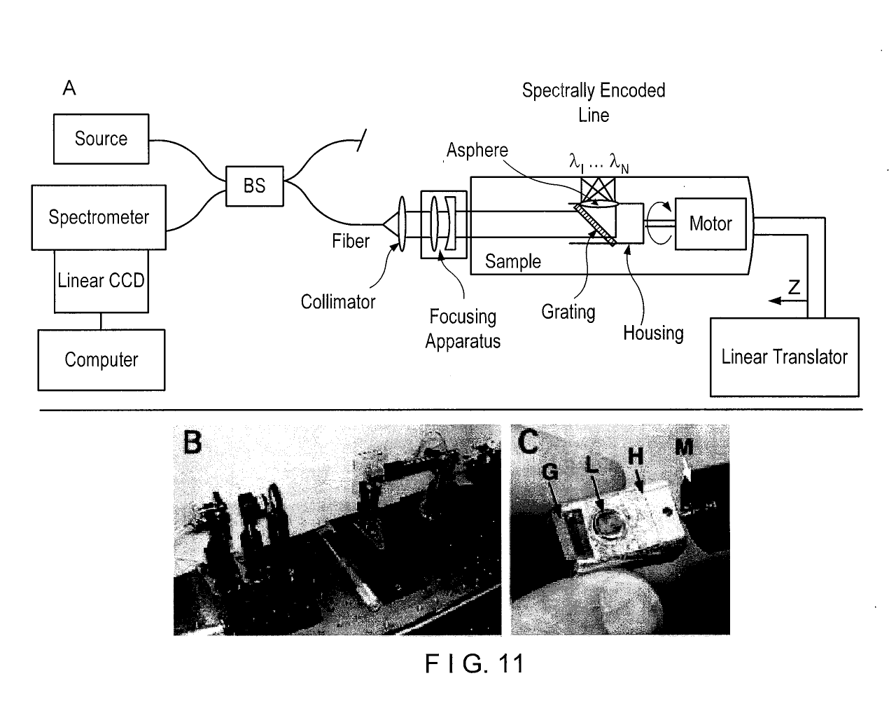

An exemplary embodiment of a probe according to the present disclosure can be provided to facilitate a comprehensive SECM imaging of the distal esophagus. With such exemplary probe (e.g., as shown in FIGS. 11A and 11B), a fiber-coupled 2.0 mW superluminescent diode can be provided, which is centered at 800 nm, and with a bandwidth of 45 nm illuminated a 50/50 single-mode fiber optic beam splitter. Light transmitted through one port of the splitter can be collimated and transmitted through a focusing apparatus to a grating-lens pair (e.g., lens −350230-B asphere, f=4.5 mm, clear aperture=5.0 mm, NA=0.55), thus producing a 500 μm longitudinal linear array of focused, spectrally-encoded spots on the interior surface of the cylindrical sample. An exemplary grating-lens pair can be affixed to a motor (e.g., 1516SR, 15 mm diameter) shaft by a custom-machined housing. As the motor rotated, the spectrally encoded line was scanned across the sample's inner circumference. Simultaneously, the motor, housing, and lens-grating pair can be translated along the longitudinal axis (z) of the cylinder by a stage, producing a helical scan of the entire interior surface of the sample. Light reflected from the sample, was transmitted back through the optical system into the single-mode fiber and delivered by the fiber to a custom-built spectrometer and linear CCD (2048 pixels, 30 kHz line rate). To achieve 1.0 μm circumferential sampling, e.g., approximately 60,000 points per motor rotation (0.5 Hz or 30 rpm) can be digitized. The longitudinal velocity of the motor can be about 0.25 mm/s. The time utilized for one complete scan of about 2.0 cm (diameter)×2.5 cm (length) area can be 100 seconds.

An exemplary 1/e² diameter of the collimated beam on the grating-lens pair can be 4.0 mm. As a result, the effective NA of the exemplary system/apparatus can be approximately 0.4, thus producing an exemplary possible spot diameter of approximately 1.2 μm, and a confocal parameter of approximately 2.5 μm. The measured transverse line spread function full-width-half-maximum (FWHM) and axial FWHM from a mirror scanned through the focus can be measured to be approximately 2.1 μm and approximately 5.5 μm, respectively. The field of view can be approximately 500 μm. These exemplary measurements can be slightly lower than theoretical values, likely due to aberrations in the optical path.

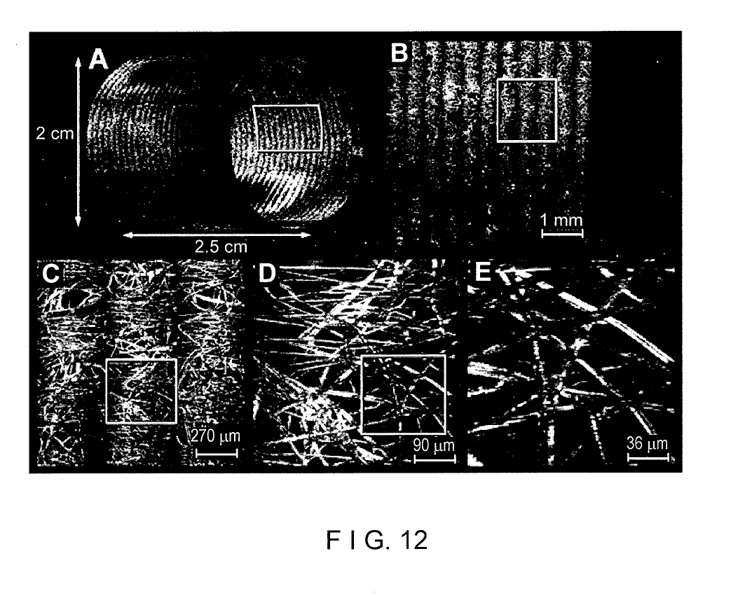

FIGS. 12A-12E illustrate exemplary SECM images for a complete pullback image of, e.g., a 2.5 cm long phantom. The exemplary phantom can consist of lens paper affixed to the inner surface of approximately 2.1 cm inner diameter Teflon tube. Cylindrical coordinates can be converted to Cartesian coordinates prior to display. At low magnification (as shown in FIG. 12A), the macroscopic structure of the paper, including folds and voids, can be visualized. When regions of this data set are shown at higher magnifications, individual fibers and fiber microstructure were clearly resolved (as illustrated in FIGS. 12B-12E).

### Exemplary Image Interpretation Procedures, Techniques and Methods

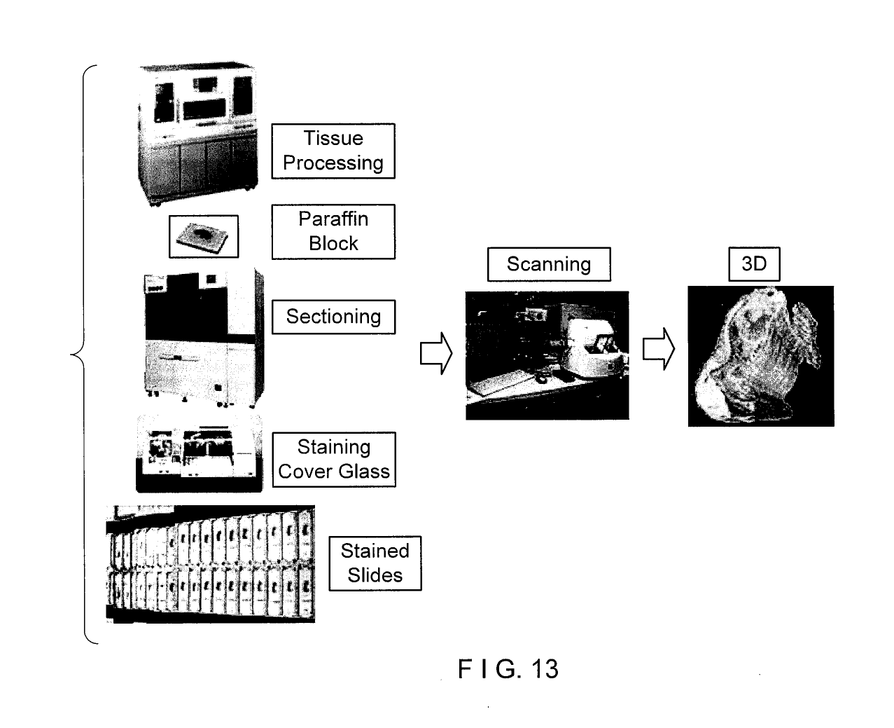

Exemplary embodiments for qualitative identification of eosinophils and automatic quantification of eosinophils can include an identification based on signal strength (as shown in FIGS. 5A-7F) and conventional histologic features (e.g., size of cell, nature of cytoplasm, and nuclear morphology). Quantitative morphometric criteria can be based on scattering intensity, cell size, and nuclear metrics developed for automated histopathology and cytopathology. Exemplary segmentation can be conducted based on these parameters, as shown in FIG. 13, and cells can be counted using conventional blob counting methods. The use of multiple depth planes may be used to optimize the robustness of the counting method. If other inflammatory cells are identified in the SECM images (e.g. mast cells, lymphocytes, neutrophils, etc.), criteria based on signal strength and nuclear morphology may also be used to identify these cell types. In addition to inflammatory cells, other histopathologic features characteristic of EoE can also be evaluated and quantitated including basal cell hyperplasia, microabscesses, degranulation, and lamina propria fibrosis.¹⁶ These features can be assembled to form a scoring system for EoE diagnosis. For example, as shown in FIG. 13, according to one exemplary embodiment of the present disclosure, exemplary computers can be used to process tissues. Thereafter, a paraffin blocking can be initiated. Further, the sectioning of the images can be performed. This can be performed by sectioning the generated images, which can be provided in three-dimensional format. The cover glasses can be stained, and the stained slides can be stored.

Transverse Vs. Cross-Sectional Image Planes

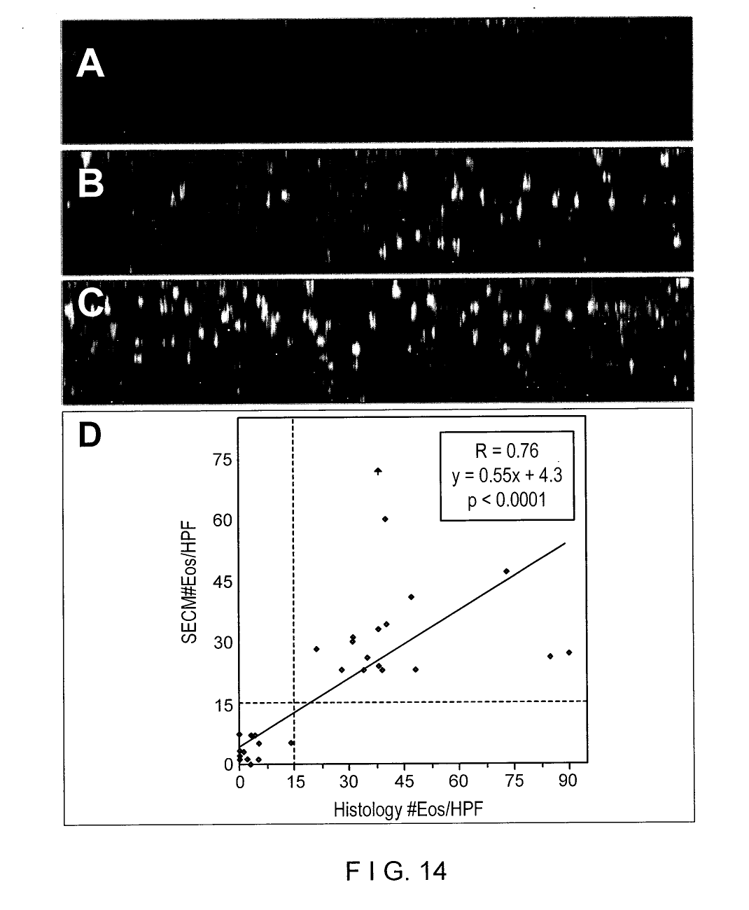

Generally, pathologists can view biopsies in a cross-sectional orientation. It is possible to obtain SECM and histology images and/or data along the transverse aspect of the biopsy, which can be perpendicular to the cross-sectional plane, as shown in FIGS. 14A-14C. Since multiple transverse sections can be obtained as a function of depth into the sample, it is possible to reconstruct cross-sectional SECM and histology images. In one exemplary embodiment of the present disclosure, this exemplary cross-sectional field can be of the same transverse dimension as a histological high power field (e.g., HPF, 40×). In an exemplary implementation of the exemplary embodiment of the present disclosure, it is possible to compare SECM eosinophil counts obtained from biopsies to histopathological eosinophil counts and found a high degree of correlation between the two methods (see FIG. 14D) and a high accuracy for diagnosis of EoE based on a eosinophil count cutoff value of greater than about 15 eos/HPF.

### Exemplary Transnasal SECM Device and Apparatus

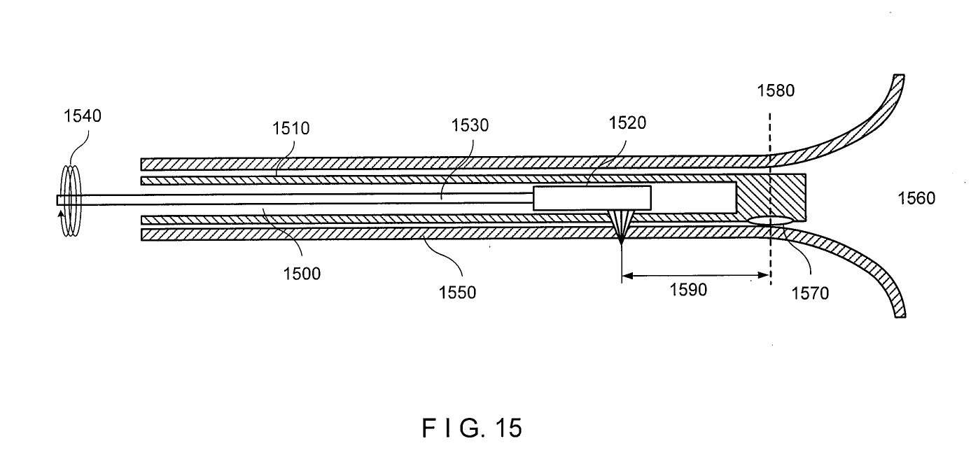

A schematic diagram of the transnasal device according to an exemplary embodiment of the present disclosure is shown in FIG. 15. The exemplary device can includes an SECM imaging probe 1530 that can be provided within a main lumen 1500 of a transparent pressure-sensitive nasogastric (PS-NG) tube 1510. The exemplary SECM probe 1530 can include an optical fiber within an inner torque-conveying cable 1520, and distal imaging optics 1520. The PS-NG tube 1510 can be dimensionally and mechanically identical or similar to a standard 10F (3.3 mm OD) NG tube. This exemplary outer diameter can be used because it is small enough to be used in the adult and pediatric populations without discomfort. In order to minimize the complexity of the exemplary probe, according to one exemplary embodiment, the exemplary device can exclude a centering balloon and can perform helical scanning 1540 in an uninsufflated esophagus 1550 (shown in FIGS. 8A, 9C). Based on the exemplary diameter of the PS-NG tube 1510, this exemplary configuration can facilitate approximately 10% of the total esophageal surface area of interest to be imaged (e.g., FIG. 9C).

The exemplary PS-NG tube 1510 and the exemplary SECM probe 1520 can be advanced to a stomach 1560 using standard NG tube placement techniques as described herein. Following a confirmation that the device is in the stomach 1560, the exemplary device can be withdrawn while recording continuous pressure measurements with a pressure sensor 1570, in a manner identical or similar to that of single-sensor esophageal manometry. The target location can be the lower esophageal sphincter (LES) 1580. Once the LES 1580 is identified, the exemplary SECM probe 1520 can be positioned ˜5 cm 1590 proximal to the LES 1580, and SECM imaging will commence. Following the exemplary imaging procedure, the entire device can be removed from the patient. This exemplary device can be cost effective, as the PS-NG tube can be sterilized and reused. If the PS-NG tube 1510 is closed, the SECM probe may likely not contact the patient and can therefore be reused without requiring sterilization between cases.

### Exemplary Pressure Measurement

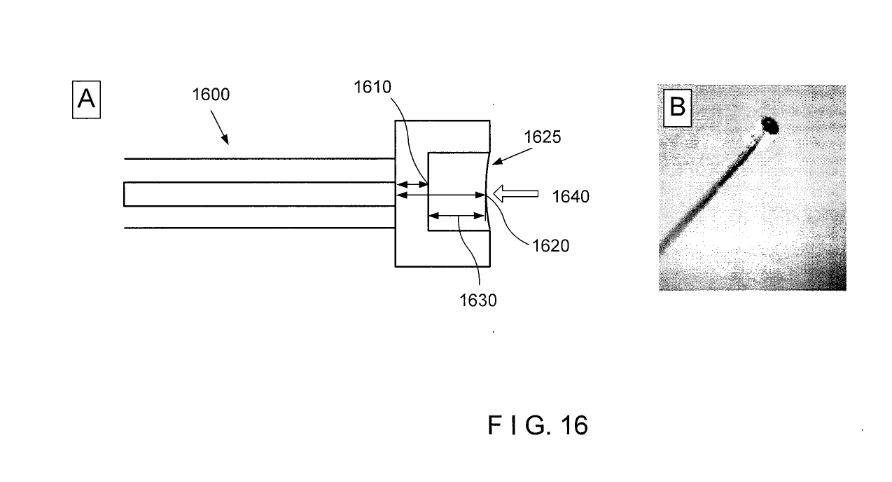

There can be different types of pressure sensors that may be incorporated into the wall of the PS-NG tube, including hydraulic, solid state, piezoresistive, and optical sensors. It can be preferable to use optical pressure sensors, as they can be extremely small, are accurate and robust, and will not require delivery of fluid or electrical current through the PS-NG tube. One exemplary optical pressure sensor is a Fabry-Perot diaphragm filter placed at the tip of an optical fiber 1600, as shown in FIG. 16. Interference between a first reflection 1610 and a second reflection 1620 from a diaphragm 1625 can be detected using white light or low-coherence interferometry. The frequency of the spectrally-resolved interference fringes can facilitate the determination of the width of the cavity, Lₛ 1630, which can then be used to determine the pressure 1640.

This exemplary pressure apparatus can comprises a 125-μm diameter single- or multi-mode optical fiber with a 250-μm diameter Fabry-Perot diaphragm filter at the tip, as shown in FIG. 16B. The exemplary sensor can measure a range of pressures (e.g., 0-50 mm Hg) which can be preferable for this exemplary application. A 250-μm buffer and/or a 325-μm polyimide coating can protect the fiber; the diaphragm 1570 can be protected by silicone. It is possible to incorporate a fiber into a smaller lumen of a custom-extruded dual lumen PS-NG tube 1510, as shown in FIG. 15. The PS-NG tube 1510 can be made from polyethylene, PVC, or silicone, and will have the same physical and mechanical characteristics of commercially available NG tubes. The PS-NG tube can be optically-transparent for the spectral region that can be used for SECM imaging. At the proximal end, a long patch cord can be extend from the pressure sensor fiber, in tandem with the SECM fiber, and can be connected to an exemplary system console via a custom-designed bulkhead connector. The exemplary system console can contain a pressure-sensing module, which includes the light source, interferometer and detector An exemplary module can have a pressure measurement resolution of 0.5 mmHg, which can be sufficient given that a ˜10 mm Hg gradient from the LES to the stomach.

### Exemplary Description of SECM Probe Design

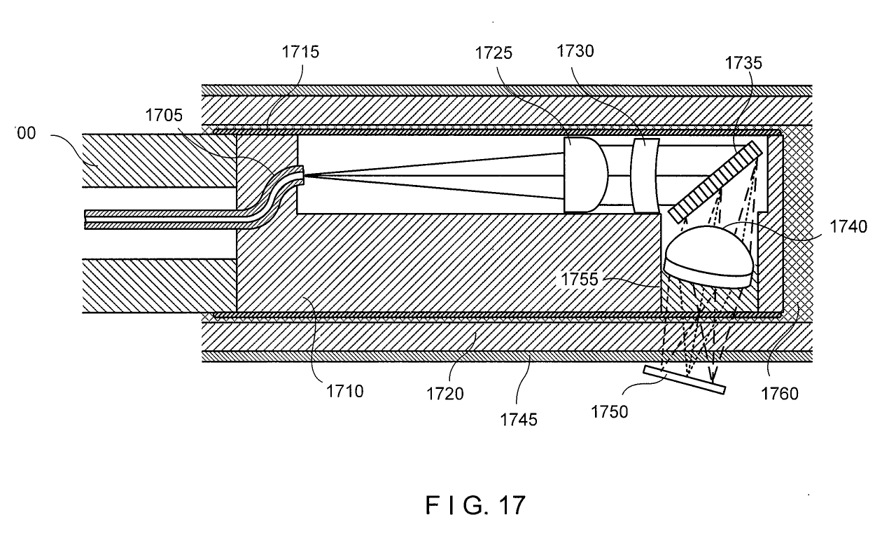

A detailed schematic diagram of an exemplary embodiment of the SECM probe according to the present disclosure is shown in FIG. 17. For example, an inner wound cable 1700 can enclose a double-clad fiber (DCF) 1705 that transceives the imaging light. In order to reduce speckle noise, imaging can be accomplished by illuminating the sample through a core of the DCF 1705 and receiving the remitted light from the sample through the inner cladding of the DCF 1705. The inner wound cable 1700 can be attached to a mechanical housing 1710 that can contain the distal optics. The mechanical housing 1710 can be enclosed by a thin, transparent sheath 1715 that can isolate the optics from the lumen of the PS-NG tube 1720. Rotating and translating the wound cable 1700 at its proximal end can facilitate helical imaging to take place over a length of approximately 10 cm. At the proximal end of the exemplary SECM probe, the wound cable 1700 can interface with the console using a rotary junction that is described in prior publications.

Within the housing 1715, the illumination light from the DCF 1705 can be collimated by a singlet lens 1725. As shown in FIG. 17 a compensation plate 1730 can be used to pre-compensate the astigmatism induced by the PS-NG tube 1720. The collimated and pre-compensated light can be diffracted by a diffraction grating 1735 (e.g., 1240 Ipmm) and focused by, e.g., a 0.46 NA objective lens 1740 onto the esophagus 1745 through the PS-NG tube 1720.

The objective lens 1740 can be assembled at an angle so that each wavelength can illuminate a distinct transverse and axial (e.g., depth) location within the esophageal wall. This exemplary configuration can facilitate a simultaneous acquisition of images at many depth locations during a single helical scan as shown in an imaging line 1750. A solid immersion layer 1755, which can comprise a plastic epoxy that has a similar refractive index to the PS-NG tube 1720, can reside between the objective lens 1740 and the inner sheath 1745. The solid immersion layer 1755 can further reduce aberrations caused by the PS-NG tube 1720. A lubricant 1760 can be used between the SECM probe's optical window and the PS-NG tube 1720 to reduce friction during the rotation and translation.

### Exemplary SECM System Console

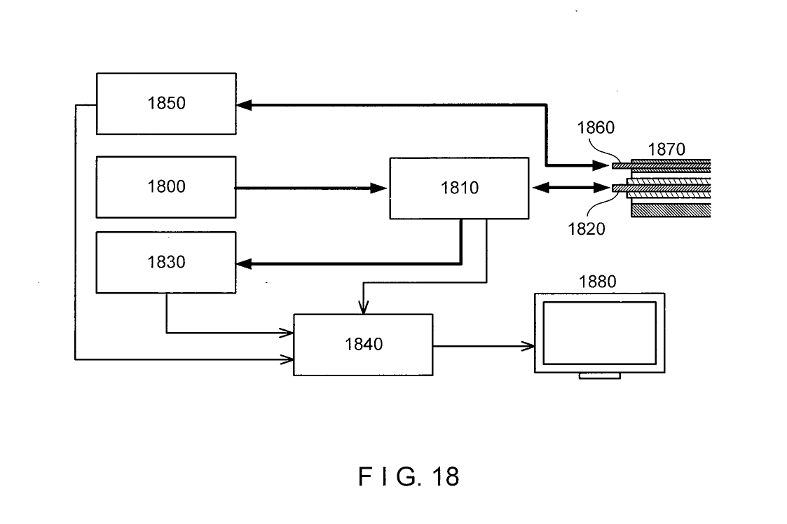

FIG. 18 shows an exemplary block diagram of an exemplary embodiment of an SECM system console according to the present disclosure. For example, such exemplary system console can contain the light sources and detectors required for SECM imaging. In one exemplary embodiment, a high-speed wavelength-swept laser 1800 (with specifications such as central wavelength=1300 nm; bandwidth=160 nm; repetition rate=254 kHz) can be utilized for imaging. Illumination light from the laser 1800 can be directed to a rotary junction 1810 that can couple the light into the core of an SECM DCF 1820. The remitted light from the tissue can be routed from the inner cladding of the DCF 1820 to the rotary junction 1810. The light from the rotary junction 1810 can be directed through a multi-mode fiber to a high-speed In GaAs photodetector 1830. The collected data from the photo detector 1830 can be digitized and transferred to the computer at a high sampling rate and saved to a data recording system 1840 (e.g., Signatec DR350, Newport Beach, Calif.) in real-time. For pressure measurements, the sensing module 1850 can provide the illumination light to and detect the returning light from the pressure sensor through a separate pressure sensing optical fiber 1860 that resides in the wall of the PS-NG tube 1870 (FIGS. 15, 18). According to another exemplary embodiment, the exemplary system can comprise a broadband light source and a spectrometer for detection of the spectrally encoded light.

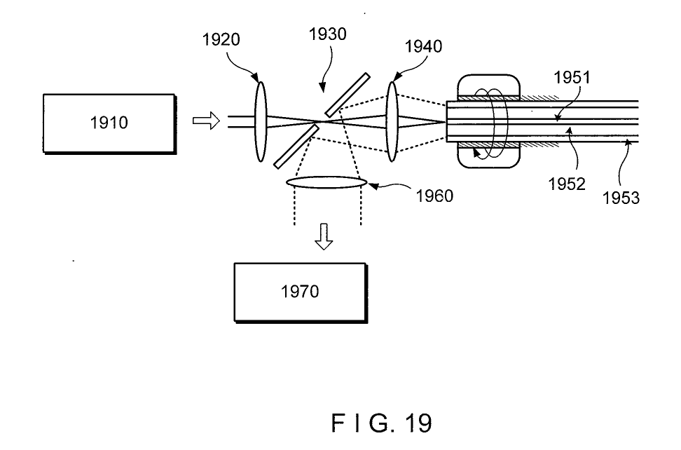

An exemplary rotary junction (as shown FIG. 19) can be provided to couple light from the console to/from the probe and rotate the SECM probe within the transparent sheath. The exemplary SECM optical rotary junction can transmit the imaging light from a light source 1910 into a core 1951 of the DCF. An inner cladding 1952 of the DCF can deliver imaging light returned from the sample to a detector 1970. In order to accomplish separation of single- from multi-mode light, the exemplary rotary junction can contain two or more focusing lenses 1920, 1940 and a single-mode/multi-mode splitter 1930, e.g., comprised of a mirror with a central transparent aperture and a relay lens (FIG. 19). The exemplary rotary junction can rotate a wound cable 1953.

### Exemplary SECM Laser

Current laser technology facilitates faster wavelength tuning over a broader bandwidth. It is possible to decrease the total time of exemplary screening procedure. Compared with prior SECM system, the exemplary embodiment of the system according to the present disclosure can be configured to image, e.g., more than fifty times faster (e.g., 254 kHz vs. 5 kHzA-line rate). In order to increase imaging speed, it is possible to provide an exemplary laser configuration for high-speed operation and will incorporate new digital data acquisition hardware. Exemplary characteristic of the scanning spectral filter of the exemplary wavelength swept lasers have been reviewed to increase the free-spectral-range of its operation while not decreasing the transmission line width. When operating the filter in this way, the laser sweep speed can be doubled and the emission duty cycle reduced by one-half. In other words, the output from the laser can be a single, fast wavelength scan followed by an equal duration interval of no emission. In order to complete the duty cycle and achieve a doubling of the repetition rate, the direct laser output can be divided into two paths having a relative delay equal to the duration of the emission. The light from the two paths can then be recombined, in a manner similar to the buffering method proposed by Huber et al. To compensate for loss in this multiplexing operation, it is possible to incorporate a buffer semiconductor optical amplifier to restore the average output power to, e.g., approximately 80 mW.

### Exemplary Specification and Performance

Table 1 depicts exemplary specifications and objective performance targets (OPTs) for the exemplary SECM probe and system. The OPTs for meeting the goals of certain exemplary embodiments of the present disclosure can be based on the a configuration of a confocal microscopy to be used for endoscopic imaging of the esophagus. The outer diameter of the transnasal probe can be approximately 3.3 mm (10F) and the rigid length approximately 10 mm, which should facilitate a convenient transnasal access for small children and adults. The longitudinal, pullback scan length of approximately 10 cm matches the typical distance between proximal and distal biopsies utilized in the standard endoscopic biopsy procedure. For an objective lens NA of, e.g., 0.46 and a DCF inner cladding mode number of, e.g., 16 in this exemplary design, it is possible to achieve a transverse resolution of approximately 1.4 μm and an axial resolution of approximately 13.5 μm, values that can be similar to those of the bench top SECM system, which was demonstrated to have a capability to visualize of eosinophils in our preliminary studies (see FIGS. 5-7). Given the tuning bandwidth of the laser source and grating-lens configuration, it is possible to obtain a total of, e.g., 236 resolvable points distributed over 10 optical sections per wavelength scan. The detector's bandwidth places an upper limit on our overall acquisition speed. Assuming, e.g., a 60 MHz detector bandwidth, a laser-tuning rate can be 254 kHz, if it is beneficial to spatially sample the esophagus at the Nyquist frequency, the dataset can comprise approximately 21 Gs. Given our 16-bit A/D sampling rate of 254 kHz×236×2 points=120 Ms/s=240 MB/s, it can therefore take, e.g., approximately 2.9 minutes to perform a complete helical scan that provides 10, 1000 mm² optical sections. If necessary, this total image acquisition duration can be reduced with certain exemplary modifications of the exemplary design.

### Exemplary Sensitivity

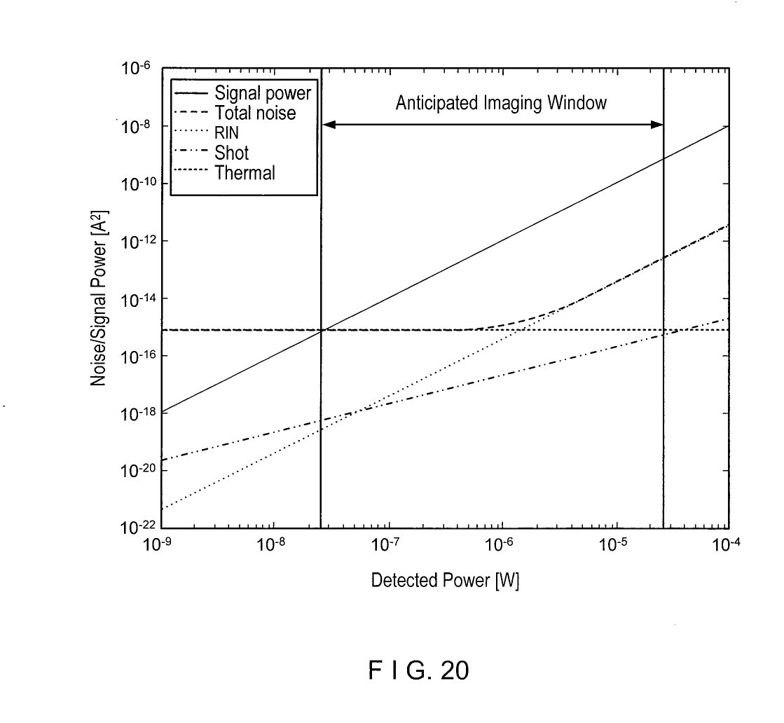

Sensitivity, defined here as the minimum detectable reflectance (SNR>1), is a key system parameter that affects image quality and penetration depth. It has been shown that, e.g., 10⁻⁴ to 10⁻⁷ of the incident light is reflected from skin at depths up to 300 μm. Since skin attenuates light more than the non-keratinized epithelial mucosa of the esophagus, and taking into account the differences in wavelengths and objective lens NA's between the two systems, it is estimated that 130 μm within tissue, the SECM probe's objective can collect approximately 3×10⁻³ to 3×10⁻⁶ of the illuminating light. Given that the exemplary source can emit, e.g., 80 mW, and an exemplary maximum 10-dB double pass insertion loss (e.g., 6 dB from probe, 4 dB from fiber optics and rotary junction), it is therefore estimates that our reflected power will range from approximately 25 μW (max) to 25 nW (min) on the detector. The noise at the detector can consist of shot noise, relative intensity noise (RIN), and thermal/electrical noise. At higher detected sample powers, the RIN noise dominates shot noise due to the narrow line width (˜0.6 nm) of our swept source.¹⁰⁵ FIG. 20 shows a plot of the noise analysis for an exemplary embodiment of the SECM system/apparatus according to the present disclosure. As can be seen from FIG. 20, SNR>1 over the entire range of remitted powers that can be expected to detect at our maximum imaging depth. If it is determined that higher sensitivity is needed, it is possible to cool the detector to reduce the thermal noise and achieve up to approximately 10 dB sensitivity improvement.

### Exemplary Components and Testing Procedures

### PS-NG Tube

For example, dual-lumen 10F (OD=3.3 mm) transnasal tubes, marked externally every cm, can be custom extruded. A variety of wall thicknesses and materials, including polyurethane, polyvinyl chloride (PVC), Tygon and silicone, can be provided to determine an appropriate combination of flexibility and maneuverability. The PS-NG tube together with the inserted SECM probe can be tested for flexibility and trackability and can be compared with commercially available NG tubes to ensure that it has the correct mechanical characteristics for transnasal or transoral insertion. It is possible to additionally test the ability of our SECM probe to be retracted within the PS-NG.

### Exemplary Pressure Sensor

Small diameter pressure sensors can comprise coated, buffered, and bare fibers can be tested for flexibility and durability. Trackability and pushability can be tested with sensors inserted into the lumen PS-NG tube. Pressure measurements can be tested and calibrated with the sensor outside the tube and inside the tube using varying, known pressures within a hermetically-sealed phantom. Different materials protecting the diaphragm can be tested for stability, sterilizability, and capability to accurately and reliably transduce external pressures.

### Exemplary Probe Optics

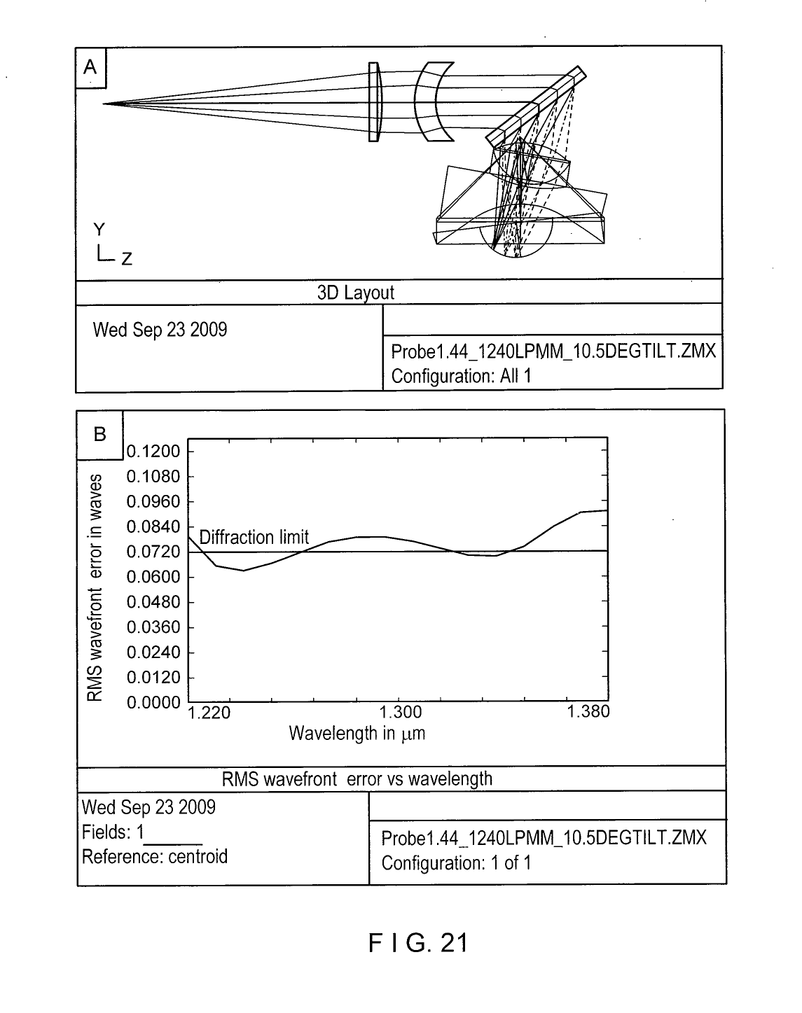

It is possible to provide exemplary optical components to miniaturize the SECM probe. In order to minimize the probe diameter and length, e.g., a collimation lens (e.g. singlet; BK7; OD=1.2 mm; f=5 mm) can be fabricated. A compensation plate (e.g. LASF35; OD=1.2 mm; f=233 mm) can be manufactured and used for the compensation of the astigmatism that is induced by non-symmetric light path through the PS-NG tube to the esophagus. A custom objective lens (doublet; LASF18 and LASF35; OD=1.2 mm; f=1.1 mm; NA=0.46) can be fabricated to have diffraction-limited optical performance for the spectral region that can be used for SECM imaging. The grating (groove density=1240 Ipmm) can be designed and fabricated to have maximum diffraction efficiency for the 1st order at the spectrum between 1220 nm to 1380 nm. It is possible to provide exemplary probe optics (FIG. 21A) in an optical modeling program/software and the simulation results demonstrate that the probe optics can have nearly diffraction-limited performance over the field of view (as shown in FIG. 21B). A miniature optical components have been previously developed with diameters of 0.5 mm. The transmission and resolution at the focal distance can be tested for the entire SECM bandwidth. Transverse resolution can be measured as the full-width-half-maximum (FWHM) of the line-spread-function (LSF) extracted from the images of a reference edge standard. The axial resolution can be measured by imaging a mirror scanned by a motorized translation stage. Spectral field of view, determined by imaging a Ronchi ruling, can be the field length over which the transverse resolution maintains its desired OPT (see Table 1).

A variety of cylindrical phantoms can be constructed to verify the probe. Exemplary resolution phantoms can consist of hollow cylinders with resolution standards affixed to the interior surface. Imaging cylindrical intralipid/gelatin phantoms and swine esophageal epithelia ex vivo can test penetration depth. Finally, segments of freshly excised swine and cadaver esophagus can be imaged using the probe.

### Exemplary Probe Assembly

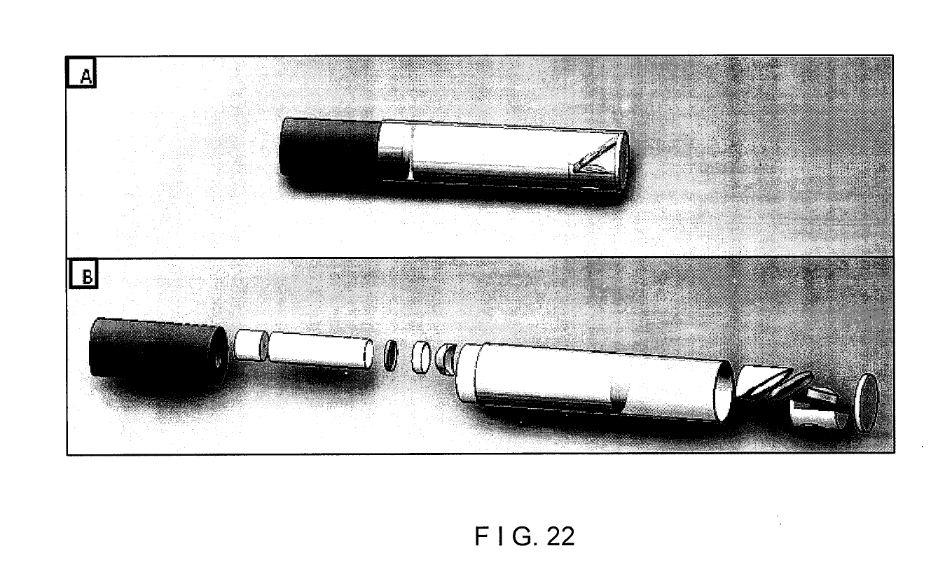

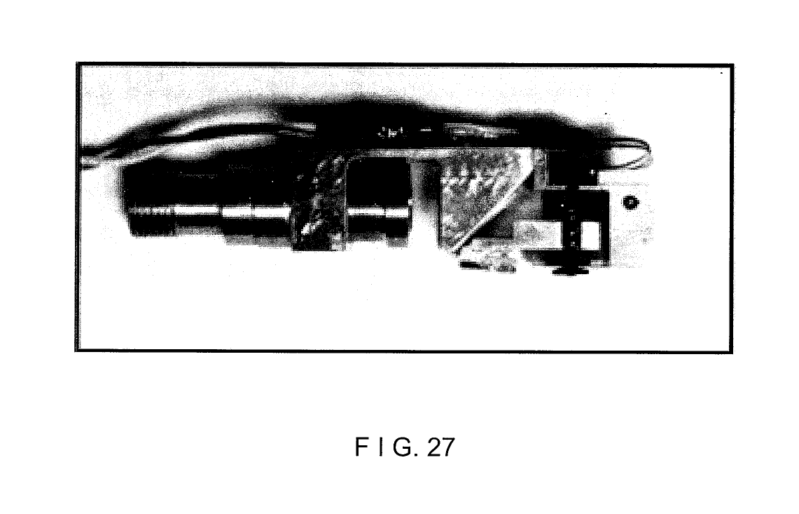

It is possible to provide an exemplary mechanical housing and the transparent sheath for the probe assembly. FIGS. 22A and 22B depict an exemplary embodiment of a probe assembly according to the present disclosure and its exploded view, respectively. The exemplary mechanical housing can be comprised of several parts for ease of fabrication. Exemplary dimensions of the parts can vary from approximately 1 mm to 7 mm, and the smallest element's size can be 125 μm. Machining of parts with these dimensions can be performed with a precision computer numerical controlled (CNC) machining system/apparatus. The exemplary optical components and the machined parts can be aligned precisely by using 3-axis, stages and assembled together using an optical-grade epoxy. One exemplary embodiment thereof is shown in FIG. 27.

DCF. Various exemplary DCFs can be provided which can include, e.g., the core-inner cladding mode ratio required to meet the axial and transverse resolution OPTs.

Wound cable. Exemplary multi-layer wound drive shafts can be used to helically scan distal optics within exemplary catheter designs. A custom wound cable can be provided for a motion transduction accuracy and repeatability through the catheter. For example, wound cables with multi-layer configurations can be provided to reduce or minimize translational and rotational distortion in the SECM images.

Lubricant. Normal saline can be used as the lubricant between the SECM probe and the PS-NG tube. Different lubricants with higher refractive indices that better match that of the PS-NG tube can be also tested to further improve the optical performance.

Probe-console interface. The optical rotary junction (FIG. 19) can be used to transmit single-mode light and detect multi-mode light transmitted through the inner cladding. The rotary junction can be designed in mechanical modeling programs and simulated in optical modeling software. Exemplary configurations can be optimized for maximum throughput and ease of manufacturing and tolerancing. The selected exemplary configuration can be provided for single and double-passed throughput and rotational uniformity. The rotary junction can additionally be provided to fit within our standard motorized pull back trays.

Exemplary Console. An exemplary embodiment of an imaging laser can be configured for power, spectrum, instantaneous line width, and repetition rate. The exemplary optics can be provided for appropriate throughput and efficiency. The optical layout can be assembled on a small breadboard for incorporation into the cart.

Software. Software can be provided to control the rotary junction and read and display the data from the pressure sensors. For example, Signatec PDA16 data acquisition software can be programmed to control the new boards and process the digitized data into images. Software can be provided for real time display of the pressure sensor's reading and integrated into the graphical user interface. Multi-resolution SECM image navigation and automated eosinophil counting can be incorporated into the existing code and user interface.

Sterilization and reuse. It is possible to use the exemplary embodiment of transnasal probe configuration according to the present disclosure so that they can be sterilized and reused, which will lower the cost of the exemplary device/apparatus, and thus the exemplary monitoring procedure. For example, it is possible to keep the external surface smooth and free of defects and will utilize materials that are stable following CIDEX or ETO sterilization procedures.

### Exemplary Alternative Embodiments and Approaches

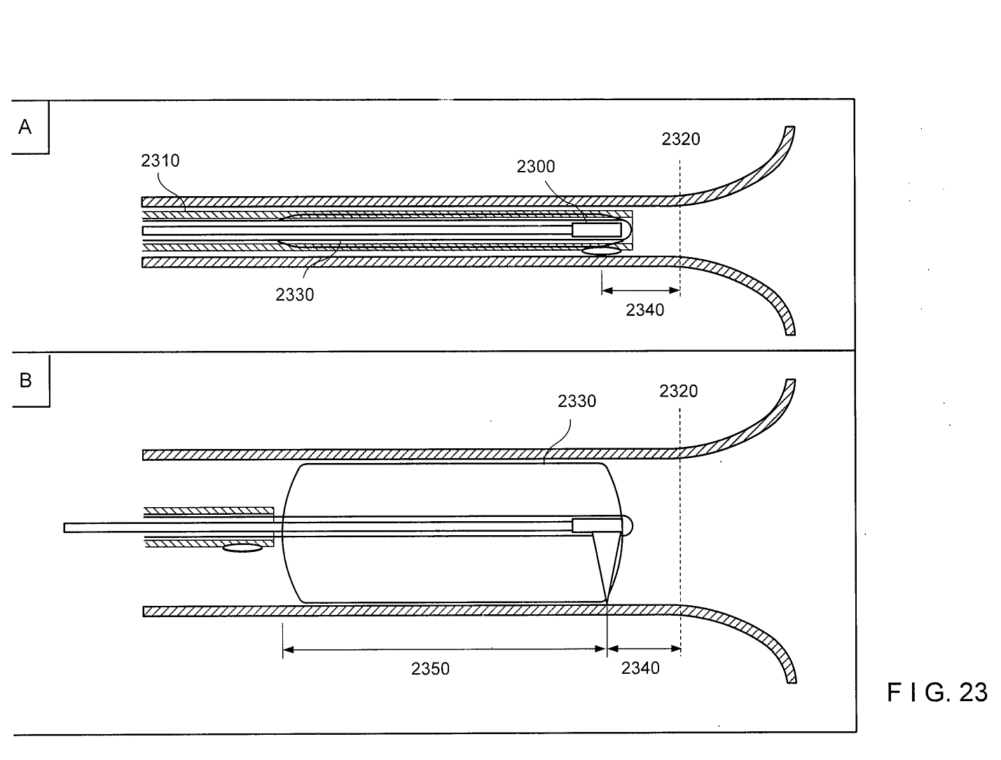

For the certain patients in which transnasal procedures are contraindicated, the PS-NG tube can be placed transorally. For safety reasons, it is can be preferable for the NG tube to not enter the airways. The exemplary approach for verifying the position of the tube can include finding an acidic pH from an aspirate. It is possible to provide another lumen in the wall of the PS-NG tube for fluid aspiration. Alternatively, it is possible to make the PS-NG tube open at the distal end, and use the SECM lumen to conduct the fluid aspiration. In this case, it is possible to either use additional sheath between the SECM probe and the PS-NG tube or make the SECM probe sterilizable. Depending on the dimension and shape of the esophagus, according to one exemplary embodiment, a contact may not be maintained between the PS-NG tube and the esophageal wall for a portion of the imaging area. An alternative approach can be to provide a balloon-centering SECM catheter and introduce the catheter through the PS-NG tube for imaging (e.g., see FIGS. 8C and 8D). For example, balloon-centering esophageal catheters have been demonstrated in exemplary OFDI clinical studies (see FIGS. 8D and 10B). FIGS. 23A and 23B illustrate schematic diagrams of an exemplary embodiment of a transnasal balloon-centering SECM catheter design and its mode of operation according to the present disclosure. As shown in these drawings, a small-diameter (e.g., 2.5 mm) balloon-centering SECM catheter 2300 can be introduced through the PS-NG tube 2310 (OD=3.3mm). When a PS-NG tube 2310 is positioned 5 cm 2340 proximal to the LES 2320 (see FIG. 23A), it can be retracted by about 10 cm 2350, exposing a balloon 2330 (FIG. 23B). The balloon 2330 can be inflated (FIG. 23B) and SECM imaging can be conducted over a length of 10 cm. Following imaging, the balloon 2330 can be deflated, the balloon catheter withdrawn into the PS-NG tube 2300, and the entire exemplary device removed from the patient.

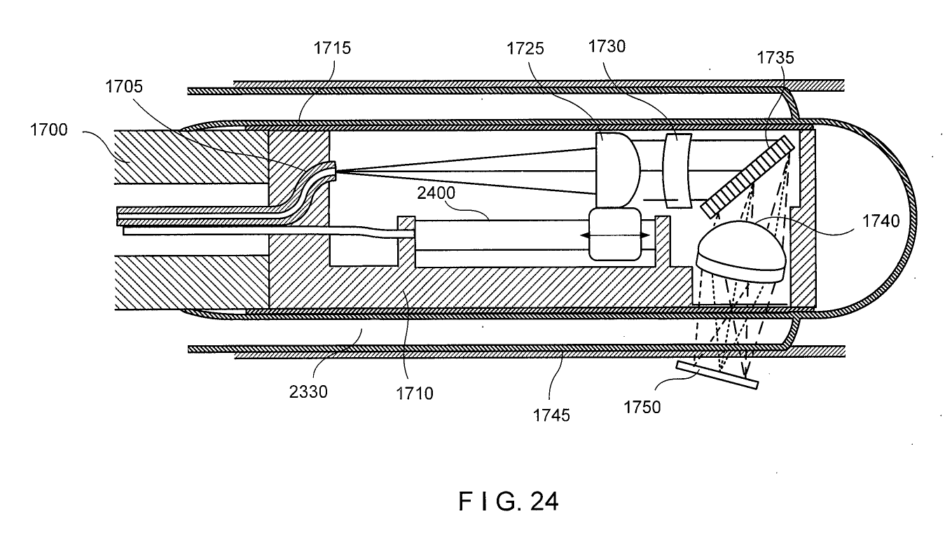

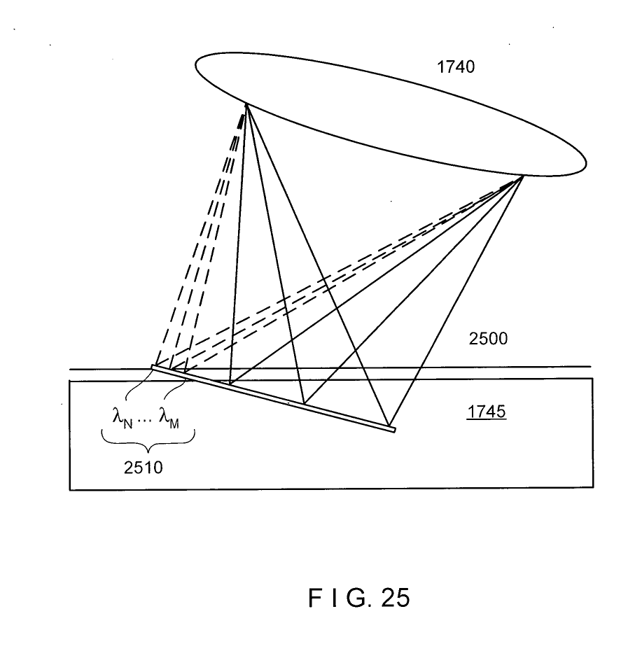

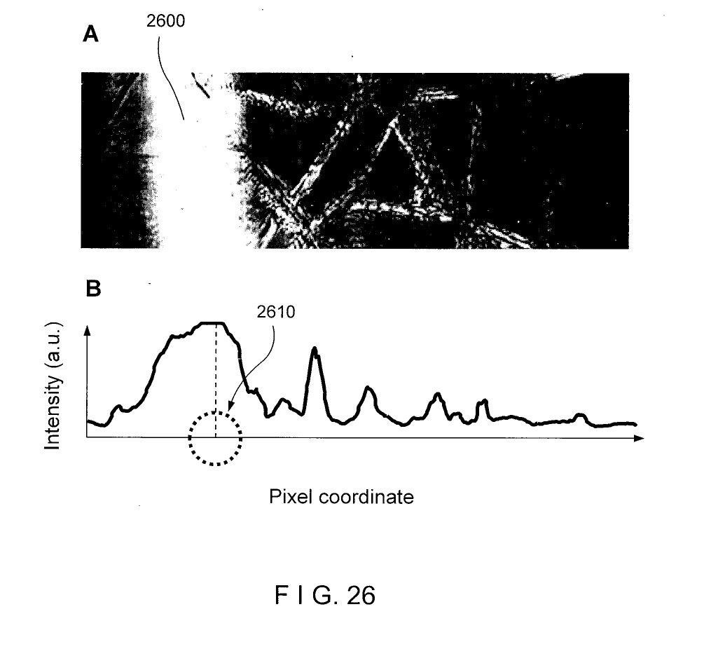

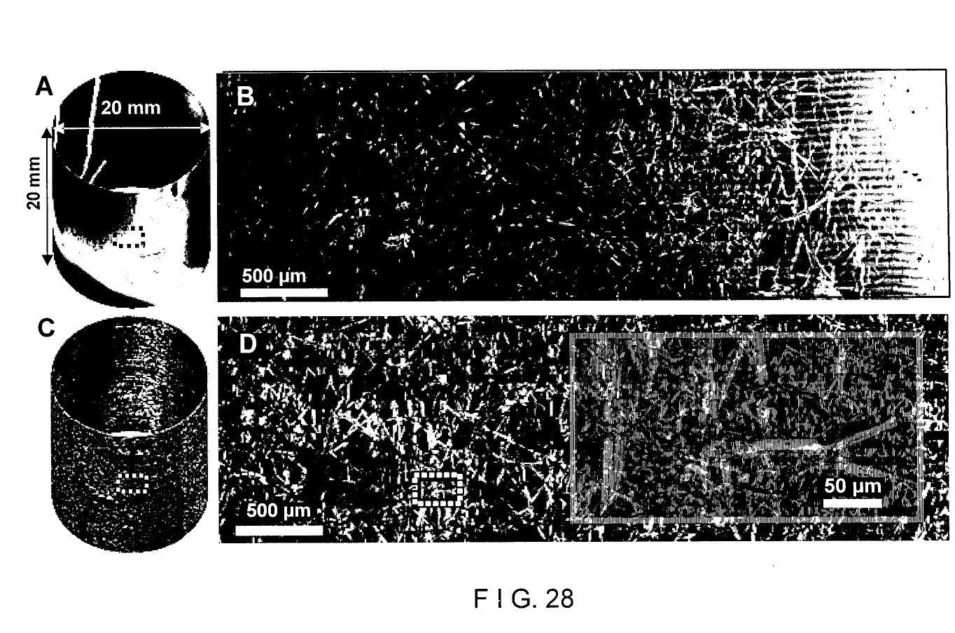

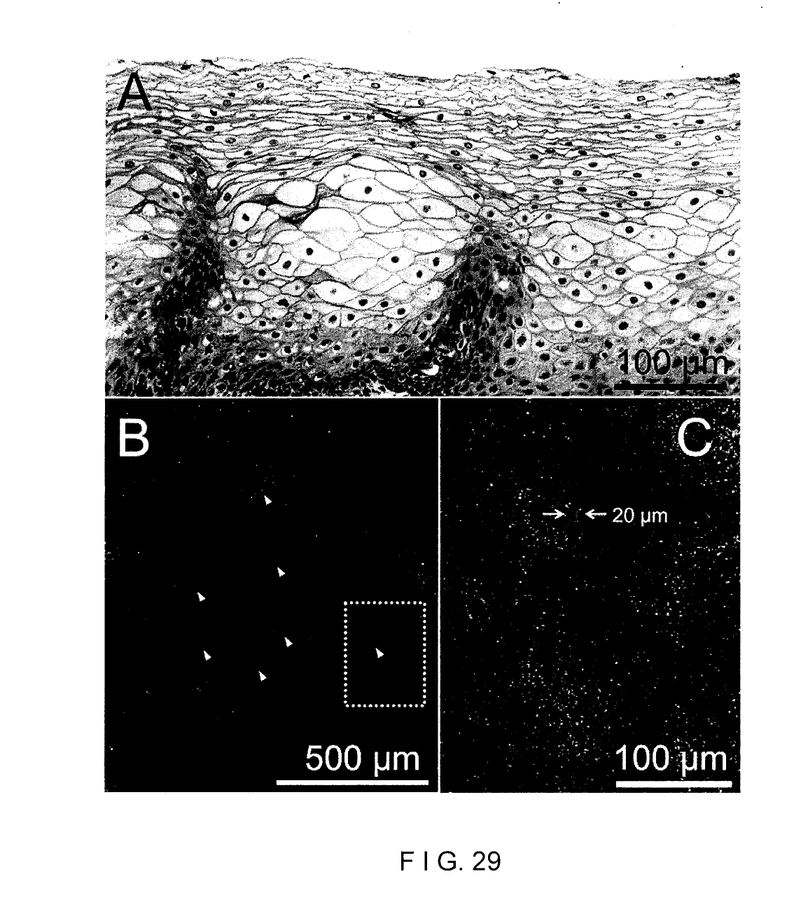

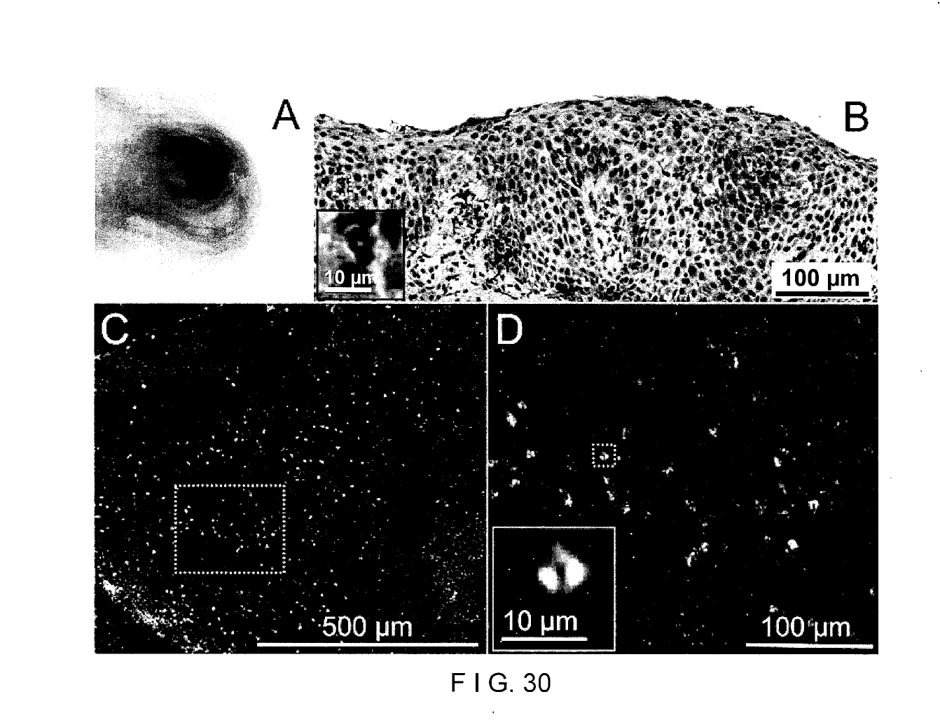

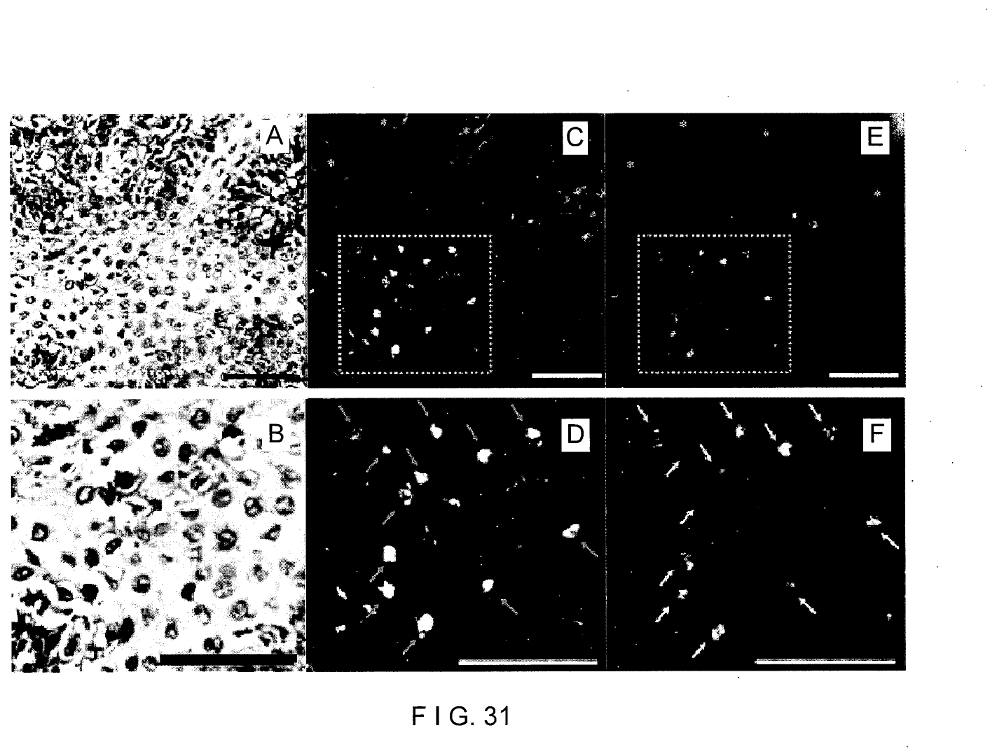

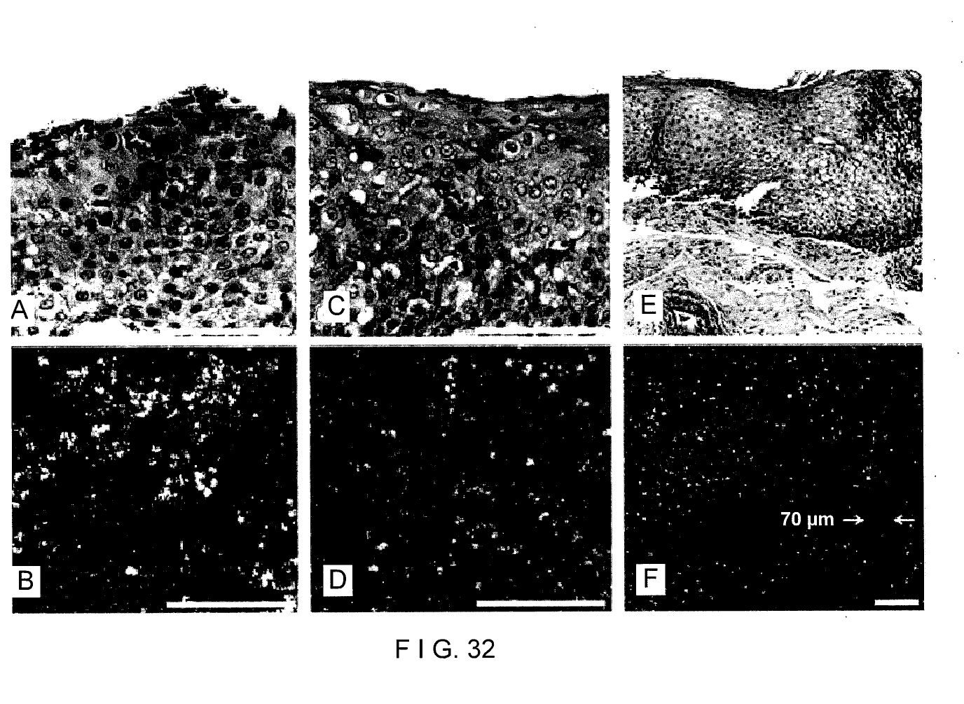

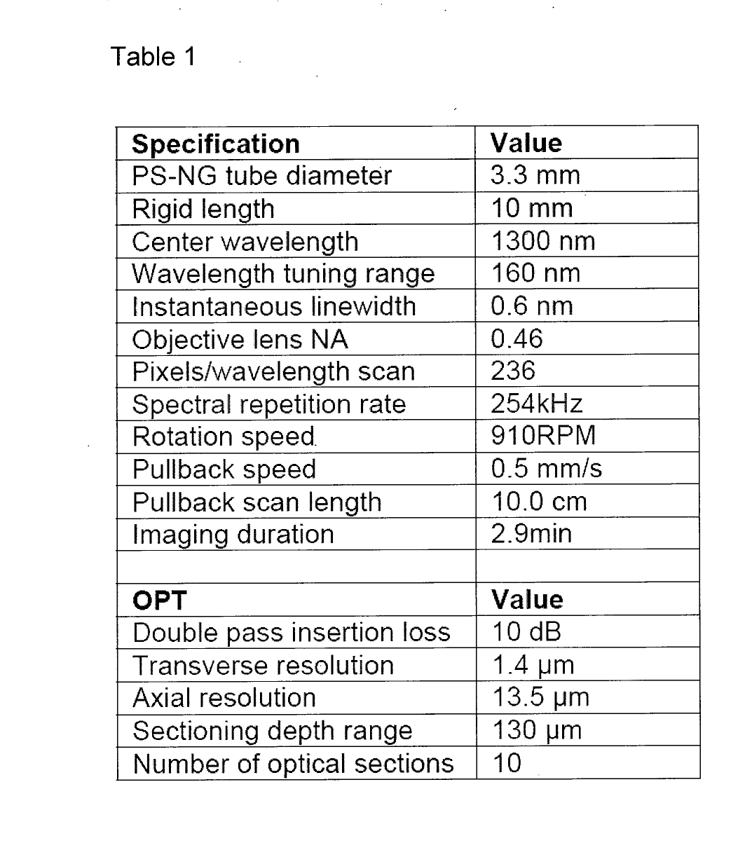

The optics of the balloon-centering SECM probe can be similar to that of the existing design with the exception that an autofocusing mechanism can be added (FIG. 24) to keep the focus within the esophageal wall during the helical scan. To conduct adaptive focusing, the balloon-centering SECM probe can have a miniature translational actuator 2400 that moves the collimation lens 1725 (FIG. 24) or the objective lens itself (FIG. 27). The displacement of the lens can change the focal plane 1750 along the axial direction. A portion of the spectrally encoded line 2510 can be used to image the bottom surface of the balloon 2500 (FIGS. 25, 26), which can be configured to have a strong reflection 2600, given by the refractive index of the balloon material. The feedback signal for controlling the focusing mechanism can be derived by determining the wavelength at which the SECM signal intensity from the balloon surface is at a maximum 2610 (FIGS. 25, 26). An exemplary miniature piezoelectric transducer actuator (shaft diameter=1 mm; moving part diameter=1.6 mm) can be provided, as well as the adaptive focusing scheme in a bench top exemplary SECM probe (FIG. 27). FIG. 28 shows exemplary SECM image data for a complete pullback image of a 2.0 cm phantom without adaptive focusing (FIGS. 28A,B) and with adaptive focusing on (FIGS. 28C,D). The phantom consisted of lens paper affixed to the outer surface of the balloon (diameter=20 mm). The exemplary SECM probe was scanned using a rotation rate of 20 rpm; a total of 400 circumferential scans were acquired in 20 minutes, limited primarily by the speed of the method used to generate the control signal. Since the length of a single spectrally-encoded line was 400 pm, the longitudinal step size of 50 μm provided 8 different depth levels. At low magnification (FIGS. 28A,C), the macroscopic structure of the paper, including folds and voids, can be visualized. When regions of this data set are shown at higher magnifications, individual fibers and fiber microstructure can be clearly resolved (FIGS. 28B,D and FIG. 28D, inset). By utilizing the automatic focusing mechanism (FIGS. 28C,D), the entire dataset remained in focus and information was acquired from all optical sections within the 50 μm range, even when the probe was not centered. In contrast, when the focusing mechanism was off, only small portions of the phantom were in focus and visible (FIGS. 28A,B). These exemplary results demonstrate the technical feasibility of comprehensive exemplary SECM for luminal organs.

Exemplary Miniaturization. It is possible to stamp an epoxy-based grating on an angle-polished cylinder

Motion artifacts. If significant longitudinal motion during sequential circumferential scans can be encountered, the scans may not line up. It is possible to correct for this occurrence by using cross-correlation techniques on adjacent scans prior to reconstructing the SECM dataset.

Exemplary Imaging penetration depth. The ranging depth of the SECM probe can be, e.g., about 130 μm. Since the eosinophilic infiltrate of EoE can typically manifests near the luminal surface, is likely that images obtained over this depth will provide sufficient information regarding the distribution of eosinophils to make a correct diagnosis. If it is determined that larger imaging penetration depth is preferred, it is possible to modify the exemplary design to increase the chromatic aberration of the objective lens, which will increase the ranging depth. An alternative approach can be to utilize a Fresnel objective lens. These configuration that enlarge the imaging penetration depth can also increase the number of discrete depth locations that can be imaged, possibly resulting in a prolonged acquisition time.

Exemplary Axial resolution. If cross-sectional imaging is used to render an accurate eosinophil count and multi-mode detection through the DCF may not provide high enough axial resolution to visualize eosinophils on cross-sectional reformatted images, different DCF configuration with smaller number of modes can be used. For example, decreasing the number of modes from about 16 to 10 can increase the speckle contrast only by about 6% while improving the axial resolution by 27%.

Increase of Speed of acquisition. The exemplary configuration can scan, e.g., a 1000 mm² area of the esophagus at about 10 different optical sections in about 2.9 minutes which is well tolerated by adult patients. If this imaging duration is determined to be too long, it can be reduced by decreasing the number of optical sections, critically sampling the data, or decreasing the pullback length. For example, one approach for reducing the total imaging time can be, e.g., to image two 1 cm longitudinal segments in the proximal and distal esophagus, a modification that can lower the acquisition time to approximately 30 seconds.

### Exemplary Method for Conducting Transnasal SECM for Diagnosing EoE

It is possible that that the exemplary unsedated transnasal SECM can be as accurate as endoscopic biopsy for counting esophageal eosinophils. This can be tested by comparing unsedated transnasal SECM to upper endoscopic biopsy, e.g., in 300 patients undergoing evaluation for EoE. In addition, information on patient tolerance of the exemplary SECM procedure can be obtained.

Unsedated transnasal SECM imaging. Prior to sedation and upper endoscopy, many or all patients can be imaged using the exemplary transnasal SECM probe and system described herein. An alternative embodiment according to the present disclosure can utilize a transoral probe described herein. Exemplary methods for insertion using intraesophageal pressure guidance can be similar or identical to those described herein above. When the probe is located at approximately 5 cm from the GEJ, helical SECM imaging will commence. The exemplary optics configuration within the exemplary probe can pull back in a helical scan from the distal to the proximal esophagus over a length of, e.g., about 10 cm at a rate of about 0.5 mm/second, resulting in a pullback duration of, e.g., about 2.9 minutes. The exemplary SECM data can be processed in real-time and viewed immediately after the pullback has completed to confirm that the data is of diagnostic quality. The exemplary optics configuration of the transnasal probe can be repositioned, and the exemplary imaging procedure repeated if necessary or desired. Following the exemplary imaging procedure, the exemplary transnasal probe can then be removed from the patient. Eosinophils may be identified and counter as per patient, imaging region, or one or more high power fields as per the methods described herein. Other features of EoE, including basal layer hyperplasia, degranulation, abscess, lamina propria fibrosis may be then rendered on the SECM or RCM dataset using qualitative user-applied criteria, semiautomatic, or automatic image processing arrangement. This information can be subsequently used to render a diagnosis of EoE or provide additional information regarding the diagnostic status of the patient.

Accordingly, exemplary embodiments of the present disclosure can provide a less invasive technology for monitoring esophageal eosinophils. Such advances can decrease the cost and of eosinophil monitoring and will increase patient tolerance for follow up eosinophil evaluation procedures. Further, the exemplary technology can be of utility to determine the pathophysiology and natural history disease in future research.

The foregoing merely illustrates the principles of the invention. Various modifications and alterations to the described embodiments will be apparent to those skilled in the art in view of the teachings herein. Indeed, the arrangements, systems and methods according to the exemplary embodiments of the present disclosure can be used with any OCT system, OFDI system, SD-OCT system or other imaging systems, and for example with those described in International Patent Application PCT/US2004/029148, filed Sep. 8, 2004, U.S. patent application Ser. No. 11/266,779, filed Nov. 2, 2005, and U.S. patent application Ser. No. 10/501,276, filed Jul. 9, 2004, the disclosures of which are incorporated by reference herein in their entireties. It will thus be appreciated that those skilled in the art will be able to devise numerous systems, arrangements and methods which, although not explicitly shown or described herein, embody the principles of the invention and are thus within the spirit and scope of the present disclosure. In addition, to the extent that the prior art knowledge has not been explicitly incorporated by reference herein above, it is explicitly being incorporated herein in its entirety. All publications referenced herein above are incorporated herein by reference in their entireties.

What is claimed is:

1. An apparatus comprising:
at least one first arrangement configured to direct at least one first electro-magnetic radiation to at least one portion of tissue within a body;
at least one second arrangement configured to receive at least one second electro-magnetic radiation provided from the at least portion, which is based on the at least one first electro-magnetic radiation; and
at least one third arrangement configured to differentiate at least one particular cell which is at least one of eosinophil, mast cell, basophil, monocyte or nutrophil from other cells in the at least one portion based on the at least one second electro-magnetic radiation.

2. The apparatus according to claim 1, wherein the at least one second electro-magnetic radiation is reflected from the at least one portion.

3. The apparatus according to claim 1, wherein the at least one third arrangement is configured to image the at least one particular cell.

4. The apparatus according to claim 3, wherein the at least one third arrangement is configured to image the at least one particular cell over a region of the tissue that greater than an are of 1 mm2.

5. The apparatus according to claim 3, wherein the at least one third arrangement is configured to image the at least one particular cell in three-dimensions.

6. The apparatus according to claim 1, wherein the at least one third arrangement is configured to image a cross-section of the at least one particular cell.

7. The apparatus according to claim 1, wherein the tissue is a luminal organ.

8. The apparatus according to claim 7, wherein the luminal organ is at least one of an esophagus or a pulmonary airway.

9. The apparatus according to claim 1, wherein at least one of the at least one first arrangement or the at least one second arrangement is provided in a catheter.

10. The apparatus according to claim 9, wherein the catheter is structured to be inserted to reach the tissue transorally or transnasally.

11. The apparatus according to claim 9, wherein the catheter has a cross-sectional diameter of less than 5 mm.

12. The apparatus according to claim 9, wherein the catheter includes a balloon arrangement.

13. The apparatus according to claim 12, wherein the balloon arrangement contains an auto-focusing arrangement which is configured to auto-focus on the at least one portion.

14. The apparatus according to claim 9, wherein the catheter is facilitated in a nasogastric tube.

15. The apparatus according to claim 9, wherein the catheter includes a wound cable.

16. The apparatus according to claim 9, wherein the catheter includes a further arrangement which is configured to measure pressure of the tissue within a body.

17. The apparatus according to claim 9, wherein the catheter has a portion to be inserted into the body which is substantially flexible.

18. The apparatus according to claim 1, wherein the at least one particular cell includes a plurality of particular cells, and the at least third arrangement is further configured to determine a number of the particular cells.

19. The apparatus according to claim 1, wherein the at least one particular cell includes a plurality of particular cells, and wherein the at least third arrangement is configured to automatically count a number of the particular cells.

20. The apparatus according to claim 1, wherein the at least one second arrangement is configured to receive a confocal light.

21. The apparatus according to claim 1, wherein the at least one second arrangement is configured to receive a spectrally encoded confocal light.

22. The apparatus according to claim 1, wherein the at least one second arrangement configured to receive and detect a florescent electro-magnetic radiation.

23. The apparatus according to claim 1, wherein at least one of the first arrangement or the second arrangements include a further arrangement which is configured to spectrally disperse at least one of the first electro-magnetic radiation or the second electro-magnetic radiation, respectively.

24. The apparatus according to claim 1, wherein at least one of the at least one first arrangement or the at least one second arrangement contains at least one optical fiber arrangement which has multiple wave-guiding regions.

25. The apparatus according to claim 24, wherein the at least one optical fiber arrangement includes a double-clad fiber core.

26. The apparatus according to claim 1, wherein the at least one first arrangement is configured to transmit at least one a broadband light or light whose frequency changes over time.

27. The apparatus according to claim 1, wherein the at least one third arrangement is further configured to differentiate at least one of basal layer hyperplasia, abscess, eosinophil degranulation or lamina propria fibrosis from other cells.

28. The apparatus according to claim 1, wherein the at least one particular cell is eosinophil.

29. The apparatus according to claim 1, wherein the at least one third arrangement differentiates the at least one particular cell based on at least one of (a) a strength of a signal from a cytoplasm or (ii) a shape of a nucleus of the at least one particular cell.

30. A method comprising:
at least one of receiving or detecting at least one first electro-magnetic radiation provided from at least one portion of tissue within a body which is based on the at least one second electro-magnetic radiation direct to the at least one portion; and
with a computer arrangement, differentiating at least one particular cell which is at least one of eosinophil, mast cell, basophil, monocyte or nutrophil from other cells in the at least one portion based on the at least one first electro-magnetic radiation.
